SEQUENCE LISTING (32 sequences)

SEQ ID NO: 1 - RNA, 21 nt, Artificial
  synthetic oligonucleotide
aagacugcguucgaauccaua

SEQ ID NO: 2 - RNA, 21 nt, Artificial
  synthetic oligonucleotide
uauggauucgaacgcagucuu

SEQ ID NO: 3 - RNA, 21 nt, Artificial
  synthetic oligonucleotide
auaguuacaaaaguuggcgcg

SEQ ID NO: 4 - RNA, 21 nt, Artificial
  synthetic oligonucleotide
cgcgccaacuuuuguaacuau

SEQ ID NO: 5 - RNA, 21 nt, Artificial
  synthetic oligonucleotide
guacaugaugcgauccaacau

SEQ ID NO: 6 - RNA, 21 nt, Artificial
  synthetic oligonucleotide
auguuggaucgcaucauguac

SEQ ID NO: 7 - DNA, 18 bp, Artificial
  synthetic oligonucleotide primer
ggaaggcattgctgctgg

SEQ ID NO: 8 - DNA, 18 bp, Artificial
  synthetic oligonucleotide primer
cctcaatgacatccttgg

SEQ ID NO: 9 - DNA, 24 bp, Artificial
  synthetic oligonucleotide primer
tgagcactcaggcaatcacactct

SEQ ID NO: 10 - DNA, 24 bp, Artificial
  synthetic oligonucleotide primer
agtgctgggtcccaagtcatgtat

SEQ ID NO: 11 - DNA, 24 bp, Artificial
  synthetic oligonucleotide primer
atgggccagcctggatatacaaca

SEQ ID NO: 12 - DNA, 24 bp, Artificial
  sythetic oligonucleotide primer
cacaagctgcacccaaacttcact

SEQ ID NO: 13 - DNA, 18 bp, Artificial
  synthetic oligonucleotide primer
ggaaggcattgctgctgg

SEQ ID NO: 14 - DNA, 18 bp, Artificial
  synthetic oligonucleotide primer
cctcaatgacatccttgg

SEQ ID NO: 15 - DNA, 29 bp, Artificial
  synthetic ologonucleotide primer
gcacgcgtgtttgtttggttggttggttg

SEQ ID NO: 16 - DNA, 23 bp, Artificial
  synthetic oligonucleotide primer
taactcgagatgctttgctggct

SEQ ID NO: 17 - DNA, 33 bp, Artificial
  synthetic oligonucleotide primer
gcacgcgtctctcttaattttctaaaatacacg

SEQ ID NO: 18 - DNA, 33 bp, Artificial
  synthetic oligonucleotide primer
gcctcgagcagaacagagcatgagttcatcact

SEQ ID NO: 19 - DNA, 29 bp, Artificial
  synthetic oligonucleotide primer
acacgcgtcagcccctgttgtaacatcct

SEQ ID NO: 20 - DNA, 32 bp, Artificial
  synthetic oligonucleotide primer
gcacgcgtccagatattccaaccactatgtgt

SEQ ID NO: 21 - DNA, 149 bp, Homo sapiens
tagtgatgaactcatgctctgttctgttttctcaaagctgaagtcggctaggtttgcaaagctgtgggctgagcactcaggcaatcacactctcagaaactgcggcggctctggactgcagcctcccaaggctccatgccagacaaagc

SEQ ID NO: 22 - DNA, 466 bp, Homo sapiens
ccagatattccaaccactatgtgttgatatgactctaaaaagaaaaagatttctgaaagcattttgcgaggcatttatagaagtaagagatattttcattttatcaccgcaacctccattaaaaattggagactattattcctggagacttcagaattttctttggaaatatctcccaattcaaaaggaagtctgacacagcctcataaaaatctatgtcgtaataggtgctgtgtcactgaaaaccactgaaaagacagggttaagaacacaaagtgagctgcacaccatatatggagaaacccgtttcttaaaactagtgatgaactcatgctctgttctgttttctcaaagctgaagtcggctaggtttgcaaagctgtgggctgagcactcaggcaatcacactctcagaaactgcggcggctctggactgcagcctcccaaggctccatgccagacaaagc

SEQ ID NO: 23 - DNA, 913 bp, Homo sapiens
cagcccctgttgtaacatccttgtcatcaccatctcacagctcttaccactgtaagctccttggggccagtgactgtgtttttatttctgtattactaaggtgaatatacaaggcaggaatttaatattagctagttcatttgatcctgccaaacctctgtaattaaggaagcagaaaaggtcccatagtctcattcaataggtggctgaggtttagagagttttgtatcctgtccaaggtcttacagctagttagaaacagaaactaaattctcaacttgagtcctgtattctttctactattctccctctcaatccctctctctctttctctctctctctctttctctccctctctctctctcacacacacacacacacacacacatacacgcttacatgtttctaccatcgtgaatgaattctaggtcaagtttatgatgttaaccagatattccaaccactatgtgttgatatgactctaaaaagaaaaagatttctgaaagcattttgcgaggcatttatagaagtaagagatattttcattttatcaccgcaacctccattaaaaattggagactattattcctggagacttcagaattttctttggaaatatctcccaattcaaaaggaagtctgacacagcctcataaaaatctatgtcgtaataggtgctgtgtcactgaaaaccactgaaaagacagggttaagaacacaaagtgagctgcacaccatatatggagaaacccgtttcttaaaactagtgatgaactcatgctctgttctgttttctcaaagctgaagtcggctaggtttgcaaagctgtgggctgagcactcaggcaatcacactctcagaaactgcggcggctctggactgcagcctcccaaggctccatgccagacaaagc

SEQ ID NO: 24 - DNA, 1439 bp, Homo sapiens
ctctcttaattttctaaaatacacgtgttttctaaaaccaggtctaaaccccacctacagctaaaatcctccaataacttcccatgactttggtgataaaataaaaaatcattagcagaattgagaaatccttcctgatctgactcttaacttctctcccccaagtggcatgaaagcatcatttggattctgaattccatgacaccaggtatcaagttttctaccagtcagaatcactacttatttacagaccatatttacagagggagacaatatggagaggttcttgctctattcttgccataacaccatgtcttcaatttcacaactagatatatagttcctatccctctaattcccttctcctctccagcgctttttcacttgatcaacacctagctgattctagcctcagcaaagacctcacctgctgtatgtaggccggctctgccgtttccagaatgggttaagtatgctctcttgtgtgtccatatctacctgtgctttcctatatccagatctccaacagcccctgttgtaacatccttgtcatcaccatctcacagctcttaccactgtaagctccttggggccagtgactgtgtttttatttctgtattactaaggtgaatatacaaggcaggaatttaatattagctagttcatttgatcctgccaaacctctgtaattaaggaagcagaaaaggtcccatagtctcattcaataggtggctgaggtttagagagttttgtatcctgtccaaggtcttacagctagttagaaacagaaactaaattctcaacttgagtcctgtattctttctactattctccctctcaatccctctctctctttctctctctctctctttctctccctctctctctctcacacacacacacacacacacacatacacgcttacatgtttctaccatcgtgaatgaattctaggtcaagtttatgatgttaaccagatattccaaccactatgtgttgatatgactctaaaaagaaaaagatttctgaaagcattttgcgaggcatttatagaagtaagagatattttcattttatcaccgcaacctccattaaaaattggagactattattcctggagacttcagaattttctttggaaatatctcccaattcaaaaggaagtctgacacagcctcataaaaatctatgtcgtaataggtgctgtgtcactgaaaaccactgaaaagacagggttaagaacacaaagtgagctgcacaccatatatggagaaacccgtttcttaaaactagtgatgaactcatgctctgttctgttttctcaaagctgaagtcggctaggtttgcaaagctgtgggctgagcactcaggcaatcacactctcagaaactgcggcggctctggactgcagcctcccaaggctccatgccagacaaagc

SEQ ID NO: 25 - DNA, 1961 bp, Homo sapiens
gtttgtttggttggttggttgggcttttttgagatggagtttcactcttgttgcctaggctagagtgcaatgacaatctcagtttactgcaactccgcctcccaggttcaagcaattctcctgcctcagcctcgcaagtagctgaggattacaggcatgtgccaccatgccaggctaattttggtatttctagtagagacagggtttcaccatgttggtcaggctggccttgcacaactgacctcagatgatccgccctcctcagcctcccaaagtgctgggattacaggcgtgagccaccgcgcctggcccgtatgtttgtttttaatgatggggcctcgctctgtcacccaggctggattgcagtgacctgactatagctcttcacagcctcaaattgtggggatcaagctatcctcccacctcaactttctgagtaggtggggctacaggtgtgcctggaatgtttctttactttttgtttgcattagaaatatttccccacctcattccattttccatctctcttaattttctaaaatacacgtgttttctaaaaccaggtctaaaccccacctacagctaaaatcctccaataacttcccatgactttggtgataaaataaaaaatcattagcagaattgagaaatccttcctgatctgactcttaacttctctcccccaagtggcatgaaagcatcatttggattctgaattccatgacaccaggtatcaagttttctaccagtcagaatcactacttatttacagaccatatttacagagggagacaatatggagaggttcttgctctattcttgccataacaccatgtcttcaatttcacaactagatatatagttcctatccctctaattcccttctcctctccagcgctttttcacttgatcaacacctagctgattctagcctcagcaaagacctcacctgctgtatgtaggccggctctgccgtttccagaatgggttaagtatgctctcttgtgtgtccatatctacctgtgctttcctatatccagatctccaacagcccctgttgtaacatccttgtcatcaccatctcacagctcttaccactgtaagctccttggggccagtgactgtgtttttatttctgtattactaaggtgaatatacaaggcaggaatttaatattagctagttcatttgatcctgccaaacctctgtaattaaggaagcagaaaaggtcccatagtctcattcaataggtggctgaggtttagagagttttgtatcctgtccaaggtcttacagctagttagaaacagaaactaaattctcaacttgagtcctgtattctttctactattctccctctcaatccctctctctctttctctctctctctctttctctccctctctctctctcacacacacacacacacacacacatacacgcttacatgtttctaccatcgtgaatgaattctaggtcaagtttatgatgttaaccagatattccaaccactatgtgttgatatgactctaaaaagaaaaagatttctgaaagcattttgcgaggcatttatagaagtaagagatattttcattttatcaccgcaacctccattaaaaattggagactattattcctggagacttcagaattttctttggaaatatctcccaattcaaaaggaagtctgacacagcctcataaaaatctatgtcgtaataggtgctgtgtcactgaaaaccactgaaaagacagggttaagaacacaaagtgagctgcacaccatatatggagaaacccgtttcttaaaactagtgatgaactcatgctctgttctgttttctcaaagctgaagtcggctaggtttgcaaagctgtgggctgagcactcaggcaatcacactctcagaaactgcggcggctctggactgcagcctcccaaggctccatgccagacaaagc

SEQ ID NO: 26 - DNA, 64 bp, Artificial
  synthetic oligonucleotide
gatcccccaagttccgatcagaggtgttcaagagacacctctgatcggaacttgtttttggaaa

SEQ ID NO: 27 - DNA, 64 bp, Artificial
  synthetic oligonucleotide
agcttttccaaaaacaagttccgatcagaggtgtctcttgaacacctctgatcggaacttgggg

SEQ ID NO: 28 - DNA, 64 bp, Artificial
  synthetic oligonucletide
gatccccgactgcgttcgaatccatattcaagagatatggattcgaacgcagtctttttggaaa

SEQ ID NO: 29 - DNA, 64 bp, Artificial
  synthetic oligonucleotide
agcttttccaaaaagactgcgttcgaatccatatctcttgaatatggattcgaacgcagtcggg

SEQ ID NO: 30 - protein, 17 aa, Artificial
  antigenic determinant used for immunization and affinity purification
DIENEDFIKDCVRIHNK

SEQ ID NO: 31 - RNA, 21 nt, Artificial
  synthetic oligonuleotide
aacaaguuccgaucagaggug

SEQ ID NO: 32 - RNA, 21 nt, Artificial
  synthetic oligonucleotide
caccucugaucggaacuuguu

(19) United States
(12) Patent Application Publication
(10) Pub. No.: US 2006/0216731 A1
(43) Pub. Date: Sep. 28, 2006

(54) Methods and reagents for diagnosing and treating gliomas

(75) Inventor: Chaya Brodie, Nof Ayalon (IL)

Correspondence Address: DR. MARK FRIEDMAN LTD.;C/o Bill Polkinghorn, 9003 Florin Way, Upper Marlboro, MD, 20772, US

(73) Assignee: BAR ILAN UNIVERSITY

(21) Appl. No.: 11/322,242

(22) Filed: Jan. 3, 2006

Related U.S. Application Data
(63) Continuation-in-part of application No. PCT/IL04/00574, filed on Jun. 29, 2004.
(60) Provisional application No. 60/483,643, filed on Jul. 1, 2003.

(30) Foreign Application Priority Data
Jun. 29, 2004 (WO) PCT/IL04/00574

(51) Int. Cl. C12Q 1/68; C12P 19/34

(52) U.S. Cl. 435/6; 435/91.2

(57) ABSTRACT

Methods and reagents for diagnosing and treating gliomas. The disclosed methods and reagents rely upon measurement or regulation of RTVP-1 in cells of the central nervous system. Also disclosed is an RTVP gene promoter, cells transformed with such vectors and transgenic animals with at least one exogenous copy of the promoter in their genomes are further disclosed.

### CROSS REFERENCE TO RELATED APPLICATION

This application is a continuation-in-part of PCT Patent Application No. PCT/IL04/00574 filed on 29 Jun. 2004, which claims priority on U.S. Application Ser. No. 60/483,643 filed 1 Jul. 2003.

### FIELD AND BACKGROUND OF THE INVENTION

The present invention relates to methods and reagents for diagnosing and treating gliomas and, more particularly, to methods and reagents which rely upon measurement or regulation of RTVP-1 in cells of the central nervous system. The present invention further includes an RTVP gene promoter as an isolated nucleic acid sequence which is further useful in targeted gene therapy. The scope of the invention further includes vectors that employ the disclosed RTVP gene promoter, cells transformed with such vectors and transgenic animals with at least one exogenous copy of the RTVP gene promoter in their genomes.

Tumors of the central nervous system (CNS) are the most prevalent solid neoplasms of childhood and the second leading cancer-related cause of death in adults between the ages of 15-34 years. The most frequent brain tumors are the gliomas, which can be divided into low-grades, anaplastic, and glioblastoma multiforme. Glioblastoma multiforme (GBM) is the most common brain tumor. The infiltrative growth pattern of this tumor prevents complete curative neurosurgery. In addition, tumor cells are resistant to currently available irradiation protocols, chemotherapy regimens and immunotherapy regimens. Mean survival time of patients with GBM is around 12 months.

Glioblastomas are divided into primary and secondary glioblastomas. These tumors are different in their onset and genetic alterations. Patients with primary glioblastomas are usually diagnosed in an advanced stage of their disease and they often die within months of diagnosis.

In contrast, many patients with secondary glioblastomas are initially diagnosed with low-grade astrocytomas, which then develop into anaplastic astrocytomas and eventually become glioblastomas.

The acquisition of anaplastic features in low-grade astrocytomas is largely unpredictable clinically and histopathologically, in particular with regards to the time over which these changes take place. While some astrocytomas show no change over more than 10 years following initial surgery, others show a rapid transition to malignancy within 1-2 years, the mean interval being 4-5 years. Progression of anaplastic astrocytoma to glioblastoma is more rapid, within approximately 2 years.

Malignant transformation of neoplastic astrocytes with low-grade features is a multistep process driven by sequential acquisition of genetic alterations. Indeed, glioblastomas express the greatest number of genetic changes. On the basis of different combination of TP53 mutations, LOH on chromosome 19q, 10q, 17p and EGFR amplification, two genetically different subsets of GBM have been defined. However none of the genetic aberrations previously identified is specific enough to constitute a reliable clinical tool.

For example, PTEN mutation is demonstrated in only 30% of primary GBM and in 5% of secondary GBM; EGFR gene amplification and overexpression of its protein product, which are considered to be the genetic hallmark of GBM, appear in only 40-60% of primary GBM and in only 10% of secondary GBM.

Thus, there is currently no specific molecular marker(s) for diagnosing glioblastoma multiforme and for assessing glial malignant transformation/progression.

Identification of specific molecular markers for GBM has potential utility in increasing reliability of diagnosis. Currently, diagnosis relies primarily upon pathohistological examination of biopsy material. This method has as an inherent disadvantage a strict requirement for subjective evaluation.

The ability to accurately differentiate malignant gliomas from other types of primary brain tumor at the time of initial diagnosis based on molecular markers has significant clinical applications.

This is especially true in cases where the histological differentiation of malignant glioma from other malignant brain tumors such as primitive neuroectodermal tumors (PNET), neuronal tumors or malignant mesenchymal tumors (anaplastic meningiomas) is problematic. Currently, no molecular marker useful in identifying malignant glioma is available.

Stereotactic brain biopsy for patients who have clinically and radiologically suspected malignant gliomas, can reveal “infiltrating zone of glial tumor” based on the initial histological evaluation. In such cases repeated surgery is currently indicated to establish the diagnosis before initiation of therapy. Because no molecular marker useful in identifying malignant glioma is available, patient anxiety and suffering is prolonged.

In addition, patients treated with surgery and/or radiation therapy for malignant gliomas typically suffer from radiological progression on their follow-up. The diagnosis of radiation necrosis versus recurrent tumor is essential for further neuro-oncological therapy. The histological mixture of radiation changes, reactive cells as well as neoplastic cells in the surgically obtained samples are quite frequent. In the absence of a glioma specific molecular marker, histological diagnosis often erroneously indicates low-grade glioma.

US Patent Application 20040009508 A1 filed by Thompson et al. and entitled “RTVP based compositions and methods for the treatment of prostate cancer” teaches that RTVP is downregulated in prostate cancer cells and that supplemental RTVP may be employed as a treatment for prostate cancer Thompson further teaches that “A further embodiment of the invention includes RTVP-specific promoters which modulate transcription (e.g. by differential methylation of promoter sequences) of RTVP in normal, pre-malignant and malignant cell. These promoters can be functionally coupled to anti-neoplastic genes to treat or prevent cell proliferative disorders such as, for example, tumors, prostate cancer, and metastatic disease.” However, the teachings of this application are limited to the murine RTVP-1 gene. Further, there is no enabling description of a promoter suited “to treat or prevent cell proliferative disorders” in the Thompson application.

WO 2002078642 by Sun and Gilbert relates to “Differentially-expressed and up regulated polynucleotides and polypeptide in breast cancer and their diagnostic and therapeutic uses.” Teachings of this application are strictly limited to breast cancer and do not include glioma progression. Because of the great disparity between breast tissue and neuronal tissue, one would not conclude that teaching of WO 2002078642 are relevant to glioma.

RTVP-1 is known as a glioma-specific gene with homology to other known genes and proteins such as TPX1 and the plant pathogenesis-related proteins (Rich et al. (1996) Gene. 180:125-130)

Human RTVP-1 and its murine homolog are expressed in a variety of tissues and in normal and transformed cell lines (Murphy et al. (1995) Gene. 159:131-135 and Ren et al. (2002) Mol Cell Biol. 22:3345-3357). Although RTVP-1 has been reported to be expressed exclusively in glioma cell lines as compared to other transformed cells in the CNS (Rich et al. (1996) Gene. 180:125-130) there have been no previous reports of differential expression of RTVP-1 in glial tumors with different degree of malignancy, although downregulation of human RTVP has been reported in prostate cancer relative to normal human prostrate (Ren et al. (2004) Cancer Res. 64:969-976).

There is thus a widely recognized need for, and it would be highly advantageous to have methods and reagents for diagnosing and treating gliomas and, more particularly, to methods and reagents which reply upon measurement or regulation of RTVP-1 in cells of the central nervous system devoid of the above limitations. Additional advantage may be realized from availability of an RTVP gene promoter as an isolated nucleic acid sequence, for example in targeted gene therapy employing vectors which include the RTVP gene promoter, from cells transformed with such vectors and from transgenic animals with at least one exogenous copy of the RTVP gene promoter in their genomes.

### SUMMARY OF THE INVENTION

According to one aspect of the present invention there is provided a method of diagnosing glioma in a subject. The method includes conducting a quantitative analysis of an RTVP expression level in a biological sample taken from the subject.

According to another aspect of the present invention there is provided a diagnostic kit for analysis of a biological sample removed from a subject. The kit includes: (a) reagents suitable for conducting a quantitative analysis of an RTVP expression level in the biological sample.

According to yet another aspect of the present invention there is provided a method of influencing a clinical progression of glioma in a subject. The method includes reducing a biological activity of RTVP-1 in the subject.

According to still another aspect of the present invention there is provided a pharmaceutical composition for treating glioma. The pharmaceutical composition includes as an active ingredient a physiologically effective amount of an agent which reduces a biological activity of RTVP-1 in a subject treated with the pharmaceutical composition and a physiologically acceptable carrier and excipient.

According to an additional aspect of the present invention there is provided a method of formulation of a pharmaceutical composition for treatment of glioma. The method includes combining an agent which reduces a biological activity of RTVP-1 with a physiologically acceptable carrier and excipient.

According to yet an additional aspect of the present invention there is provided an isolated nucleic acid sequence characterized by an ability to positively regulate a downstream gene. The sequence includes an item selected from the group consisting of SEQ. ID. NO.: 25 and a functional portion thereof.

According to further features in preferred embodiments of the invention described below, the quantitative analysis relies upon quantification of at least a portion of an RTVP mRNA transcript.

According to still further features in the described preferred embodiments the quantification of at least a portion of an RTVP mRNA transcript is accomplished by RT-PCR.

According to still further features in the described preferred embodiments the RT-PCR employs at least one primer selected from the group consisting of SEQ. ID. NOs.: 7; 9 and 11.

According to still further features in the described preferred embodiments the RT-PCR employs at least one primer selected from the group consisting of SEQ. ID. NOs.: 8; 10 and 12.

According to still further features in the described preferred embodiments the quantification includes a comparison to at least a portion of at least one additional transcript.

According to still further features in the described preferred embodiments the at least one additional transcript includes an S12 transcript.

According to still further features in the described preferred embodiments the quantitative analysis of an RTVP expression level in a biological sample taken from the subject employs an antibody specific to at least a portion of an RTVP-1 protein.

According to still further features in the described preferred embodiments the diagnostic kit further includes (b) packaging material; and (c) instructions for performance of the quantitative analysis on at least one type of biological sample.

According to still further features in the described preferred embodiments the instructions further include an explanation of at least one method for collection of the biological sample.

According to still further features in the described preferred embodiments the instructions further include an explanation of diagnosing glioma in the subject based upon a result of the analysis.

According to still further features in the described preferred embodiments the reducing of the biological activity is accomplished by influencing at least one item selected from the group consisting of a level of transcription of an RTVP-1 gene, a stability of an RTVP-1 mRNA transcript, a level of translation of an RTVP-1 mRNA transcript, a level of activity of an RTVP-1 protein and a stability of an RTVP-1 protein.

According to still further features in the described preferred embodiments the influencing a stability of an RTVP-1 mRNA transcript is accomplished by administration of siRNA to the subject.

According to still further features in the described preferred embodiments the siRNA includes at least one pair of complementary RNA sequences selected from the group consisting of SEQ. ID. NOs: 1 and 2; SEQ. ID. NOs: 3 and 4; SEQ. ID. NOs: 31 and 32; SEQ. ID. NOs: 26 and 27; and SEQ. ID. NOs: 28 and 29.

According to still further features in the described preferred embodiments the reducing biological activity of RTVP-1 employs an antibody specific to at least a portion of an RTVP-1 protein.

According to still further features in the described preferred embodiments the functional portion of the isolated nucleic acid sequence is selected from the group consisting of SEQ, ID. NOs.: 21; 22; 23 and 24.

According to still further features in the described preferred embodiments there is provided an expression vector that includes the isolated nucleic acid sequence of RTVP-1 characterized by an ability to positively regulate a downstream gene.

According to still further features in the described preferred embodiments there is provided a mammalian cell transfected with the expression vector described hereinabove.

According to still further features in the described preferred embodiments there is provided a transgenic animal, the transgenic animal includes at least one exogenous copy of the isolated nucleic acid sequence characterized by an ability to positively regulate a downstream gene within a genome thereof.

The present invention successfully addresses the shortcomings of the presently known configurations by providing methods and reagents for diagnosing and treating gliomas and, more particularly, to methods and reagents which rely upon measurement or regulation of RTVP-1 in cells of the central nervous system. Additional advantage may be realized from disclosed RTVP gene promoter. This promoter may find utility in targeted gene therapy employing vectors which include the RTVP gene promoter, in cells transformed with such vectors and in transgenic animals with at least one exogenous copy of the RTVP gene promoter in their genomes.

Implementation of analyses as described in the context of the present invention may include performing or completing selected tasks or steps manually, automatically, or a combination thereof. Moreover, according to actual instrumentation and equipment of preferred embodiments of the present invention, several selected steps could be implemented by hardware or by software on any operating system of any firmware or a combination thereof. For example, as hardware, selected steps of the invention could be implemented as a chip or a circuit. As software, selected steps of the invention could be implemented as a plurality of software instructions being executed by a computer using any suitable operating system. In any case, selected steps of the analysis could be described as being performed by a data processor, such as a computing platform for executing a plurality of instructions.

### BRIEF DESCRIPTION OF THE DRAWINGS

The invention is herein described, by way of example only, with reference to the accompanying drawings. With specific reference now to the drawings in detail, it is stressed that the particulars shown are by way of example and for purposes of illustrative discussion of the preferred embodiments of the present invention only, and are presented in the cause of providing what is believed to be the most useful and readily understood description of the principles and conceptual aspects of the invention. In this regard, no attempt is made to show structural details of the invention in more detail than is necessary for a fundamental understanding of the invention, the description taken with the drawings making apparent to those skilled in the art how the several forms of the invention may be embodied in practice.

In the drawings:

FIGS. 1a, 1b and 1c illustrate Ethidium Bromide stained gels which demonstrate relative transcript levels of RTVP-1 and S12 in control cells, cell treated with scrambled SiRNA (SsiRNA) and siRNA specific to RTVP(RTVP-siRNA). Panel a from U87 cells, Panel b from A172 cells and Panel c from PC3 cells

FIG. 2a is an immunoblot illustrating PCNA expression in brain, LGA and GBM. FIGS. 2b and 2c are comparative bar graphs illustrating that silencing of RTVP-1 decreased glioma cell number (FIG. 2b) and induced apoptosis (FIG. 2c) in U87 and A172 cells.

FIGS. 3a, 3b and 3c illustrate that a second siRNA (SiRTVP-1(II) ) significantly reduced the level of RTVP-1 mRNA (FIG. 3a; Ethidium Bromide gel as in FIG. 1) while a control scrambled siRNA had no effect. Cells transfected with the siRNA-RTVP-1 (II) exhibited reduced cell proliferation and increased cell apoptosis (FIGS. 3b and 3c respectively).

FIGS. 4a, 4b and 4c are similar to FIG. 3 except that a plasmid vector was employed to deliver RTVP-siRNA as in FIG. 1.

FIG. 5 is an Ethidium Bromide stained gel showing the products of RT-PCR in the following glioma cell lines: U87 (1), A172 (2), T98G (3), LN 18 (4), LN-229 (5), U251 (6) U118 (7) and in primary human astrocytes (8). The S12 mRNA RT-PCR product is used as a control.

FIGS. 6a, 6b and 6c are Ethidium Bromide stained gel showing the products of RT-PCR as in FIG. 5 on samples from adult brain (brain), low-grade astrocytomas (LGA), anaplastic astrocytomas (AA), glioblastoma multiforme (GBM) and anaplastic meningiomas (AM). The S12 RNA RT-PCR product is used as a control.

FIGS. 7a, 7b, 8a, 8b, 8c and 8d are similar to FIG. 6. Lanes 1-5 and 20-38 are GBM samples, lanes 6-10 and 50-58 are LGA samples, lanes 40-50 are AA samples and lanes 11, 59 and 60 are adult brain.

FIG. 9 is a diagram illustrating relative sizes and positions of RTVP-1 promoter fragments A, B, C, D and E.

FIG. 10a is comparative bar graph illustrating the relative effect of fragments C and D on a downstream Luciferase gene in U87 cells.

FIG. 10b is a comparative bar graph illustrating the relative effect of fragment D on a downstream Luciferase gene in Hela cells and U87.

FIG. 11 is a comparative bar graph illustrating the relative effect of fragments A,B,C, D and E on a downstream Luciferase gene in U87.

FIG. 12 is a comparative bar graph illustrating the relative expression of RTVP-1 in GBM, AA, LGA and normal brain based on the ratios of the RTVP-1 mRNA/S12 mRNA of the RT-PCR results described in FIGS. 6a; 6b; 6c; 7a; 7b; 8a; 8b; 8c; and 8d. The results are presented as the mean values ±S.E. Data were analyzed using one-way ANOVA to determine the level of significance between the different groups. (*p<0.001, **p<0-002; relative to GBM).

FIG. 13 is an immuno-blot illustrating presence of a FLAG™ expression tag in whole cell extract and culture media from U87 cells transfected with RTVP-1 in pCMVtg2b or with the empty vector (CV) for 24 hr. Medium was changed to serum free medium and after additional 24 hr, cells and cell supernatants were collected, processed and analyzed by Western blot analysis. The expressed and secreted RTVP-1 was identified with an anti-FLAG antibody (Sigma Chemical; St. Louis Mo.).

FIGS. 14a through 14e show expression of RTVP-1 in astrocytic tumors and glioma cell lines. Total RNA was extracted from normal brains, low-grade astrocytomas (LGA, grade II), anaplastic astrocytomas (AA, grade III) and glioblastomas (GBM, grade IV). The expression of RTVP-1 was determined using semi-quantitative RT-PCR (FIGS. 14a, 14b) or real-time PCR (FIG. 14c) as described in the Methods.

FIG. 14b is a bar graph showing the results of densitometry of the semi-quantitative RT-PCR (relative RTVP-1 mRNA to S12) for normal brains (n=8), LGA (n=16), AA (n=23) and GBM (n=33). (* P<0.05, ** P<0.001).

FIG. 14c shows the results of real-time PCR performed using RTVP-1 and S12 probes of mRNA from five normal brain tissues, 16 low-grade astrocytomas (LGA), 20 anaplastic astrocytomas (AA), 26 oligodendrogliomas (Oligo) and 22 glioblastomas (GBM). Results are normalized relative to the levels of S12 mRNA and are presented relative to reference sample, Mean values are marked.

FIG. 14d shows RTVP-1 levels determined using semi-quantitative RT-PCR. RNA was extracted from glioma cell lines, primary glioma cultures and normal hum-an astrocytes.

FIG. 14e shows the expression of RTVP-1 in U87 and A172 cells determined using Northern blot analysis. After hybridization, membranes were rehybridized using a GAPDH probe to control for variations in gel loading and transfer efficiency. The results are from one representative out of four similar experiments,

FIG. 15 illustrates that silencing of RTVP-1 decreases cell growth and induces cell apoptosis. FIG. 15a illustrates the expression of RTVP-1 mRNA determined after 3 days using semi-quantitative RT-PCR, where U87, A172, HF1254 and HF1308 cells were transfected with siRNAs targeting the RTVP-1 mRNA. S12 was used to control for equivalent loading/amplification.

FIG. 15b examines the effect of the RTVP-1 siRNA on the RTVP-1 protein levels in U87 cells overexpressing RTVP-1 using anti-FLAG antibody.

FIG. 15c is a bar graph illustrating the percentage of cell proliferation, where cells were counted after 3 days of siRNA transfection.

FIG. 15d is a bar graph illustrating the number of colonies. For the anchorage-independent growth of the cells, A172 and U87 cells transfected with control siRNA and with RTVP-1 siRNA were plated in soft-agar for 8 days and colonies were viewed under a phase contrast microscope and counted.

FIG. 15e is a bar graph showing the percentage of apoptotic cells. The apoptosis of the cells was determined using PI staining and FACS analysis. The results represent the means ±S.E of three independent experiments (FIGS. 15c, 15d and 15e) or represent one of four separate experiments, which gave similar results (FIGS. 15a, 15b).

FIG. 16 shows expression of GFP under the RTVP-1 promoter in U87 and MCF-7 cells. U87 and MCF-7 cells were infected with SMOI of adenovirus vector expressing the GFP protein under the RTVP-1 promoter. After 24 hours cells were viewed under a confocal microscope.

### DESCRIPTION OF THE PREFERRED EMBODIMENTS

The present invention is of methods and reagents for diagnosing and treating gliomas which can be used predict clinical severity in specific patients and/or as modes of treatment.

Specifically, the present invention can be used to measure or to regulate RTVP-1 in cells of the central nervous system. Regulation is achieved by the disclosed RTVP gene promoter(s). These promoters may find utility in targeted gene therapy, in cells transformed with such vectors including the promoter and in transgenic animals with at least one exogenous copy of the RTVP gene promoter in their genomes.

The principles and operation of methods and reagents for diagnosing and treating gliomas according to the present invention may be better understood with reference to the figures, experimental examples and accompanying descriptions.

Before explaining at least one embodiment of the invention in detail, it is to be understood that the invention is not limited in its application to the details set forth in the following description or exemplified by the Examples. The invention is capable of other embodiments or of being practiced or carried out in various ways. Also, it is to be understood that the phraseology and terminology employed herein is for the purpose of description and should not be regarded as limiting.

The term “diagnosing” as used in this specification and the accompanying claims is to be construed in its broadest possible sense so that it may include analysis of the presence/absence of glioma and/or analysis of severity of a clinical prognosis of a glioma discovered to be present.

The phrase “biological sample” as used in this specification and the accompanying claims is to be construed in its broadest possible sense so that it may include solid tissue (e.g. tumor biopsy material), viscous liquids (e.g. lymph gland contents) and biological fluids (e.g. blood, cerebrospinal fluid and ascites fluid).

The term “expression level” as used in this specification and the accompanying claims is to be construed in its broadest possible sense so that it may include a level of transcription of an RTVP mRNA, a level of stability of an RTVP mRNA, a level of translation of an RTVP mRNA to RTVP or a stability of an RTVP protein (e.g. with respect to enzymatic degradation).

The term “RT-PCR” as used in this specification and the accompanying claims refers to reverse transcriptase-polymerase chain reaction. One of ordinary skill in the art will be able to adapt this generally known technique for use in the context of the present invention using details disclosed hereinabove and hereinbelow.

The present invention is primarily embodied by a method of diagnosing glioma in a subject. The method includes conducting a quantitative analysis of an RTVP expression level in a biological sample taken from the subject.

The present invention is further embodied by a diagnostic kit for analysis of a biological sample removed from a subject. The kit includes reagents suitable for conducting a quantitative analysis of an RTVP expression level in the biological sample. In other words, the kit facilitates practice of the diagnostic method.

According to still further features in the described preferred embodiment, the diagnostic kit further includes packaging material and instructions for performance of the quantitative analysis on at least one type of biological sample. The instructions, in a most preferred embodiment, further include an explanation of at least one method for collection of the biological sample from the subject. Alternately, or additionally, the instructions further include an explanation of diagnosing glioma in the subject based upon a result of the analysis.

Optionally, but preferably, the kit further includes reagents for generation of standards for comparison, most preferably the standard for comparison is a calibration curve.

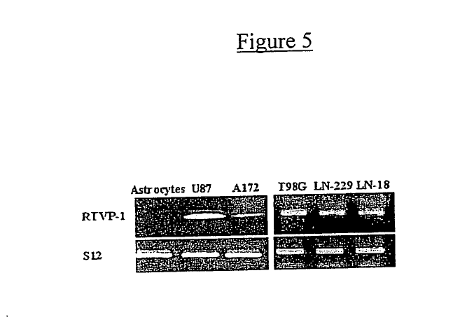

According to some preferred embodiments of the diagnostic method and the diagnostic kit, the quantitative analysis relies upon quantification of at least a portion of an RTVP mRNA transcript. Quantification of at least a portion of an RTVP mRNA transcript may be accomplished, for example, by RT-PCR (see FIGS. 5, 6a, 6b, 6c, 7a, 7b, 8a, 8b, 8c and 8d)

Quantitation by northern blot analysis is also within the scope of the invention. A positive correlation between increased severity of clinical prognosis and increased RTVP-1 transcriptional activity is demonstrated in examples 1 and 2 hereinbelow. This correlation is opposite to the correlation reported by Thompson in US Patent Application 20040009508 A1. Ren et al. (Mol. Cell Biol. 22:3345, 2002) also concluded that overexpression of RTVP-1 in prostate cancer cell lines induced cell apoptosis in contrast to results presented hereinbelow.

Forward primers suitable for use in RT-PCR in the context of the claimed diagnostic method included those identified as SEQ. ID. NOs.: 7; 9 and 11, although other forward primers might be used without significantly effecting the reliability of the diagnostic method or the utility of the diagnostic kit.

Reverse primers suitable for use in RT-PCR in the context of the claimed diagnostic method included those identified as SEQ. ID. NOs.: 8; 10 and 12, although other reverse primers might be used without significantly effecting the reliability of the diagnostic method or the utility of the diagnostic kit.

In order to control for differences between samples (e.g. level of degradation of RNA, loading, etc.) the quantification preferably includes a comparison to at least a portion of at least one additional transcript such as, for example, an S12 transcript.

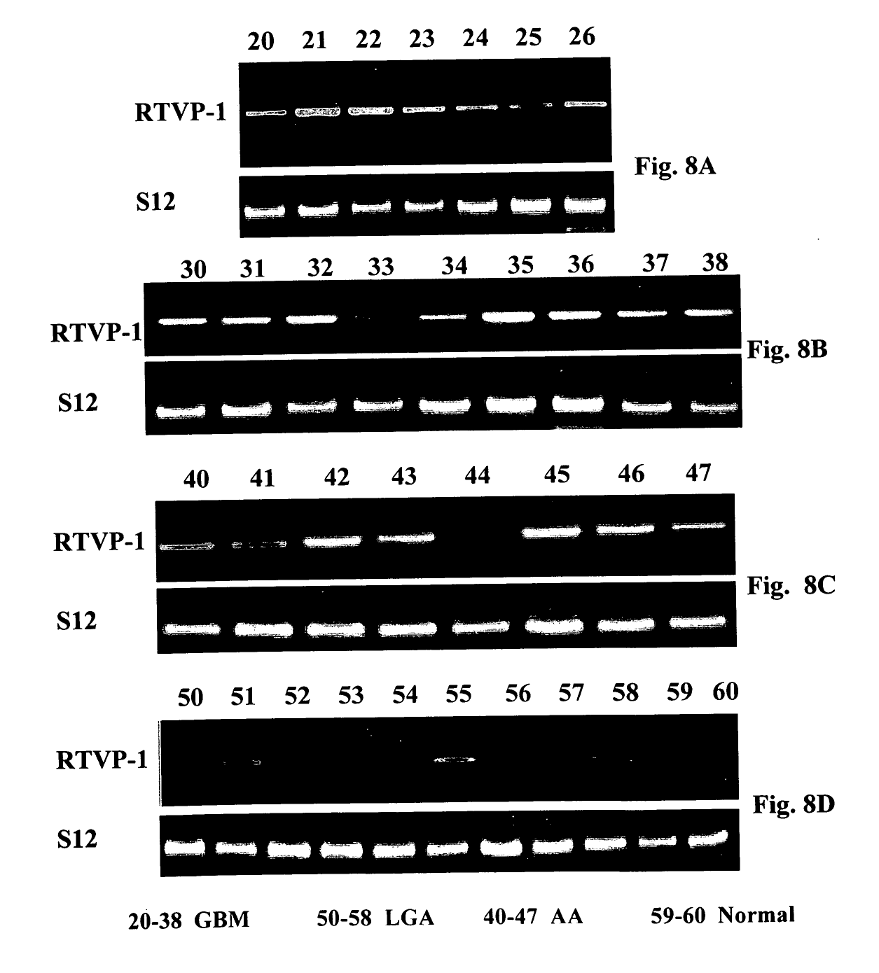

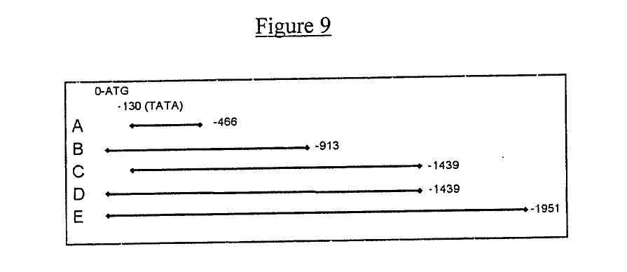

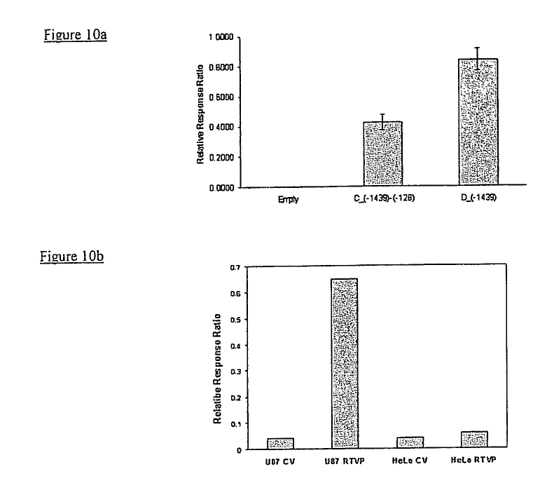

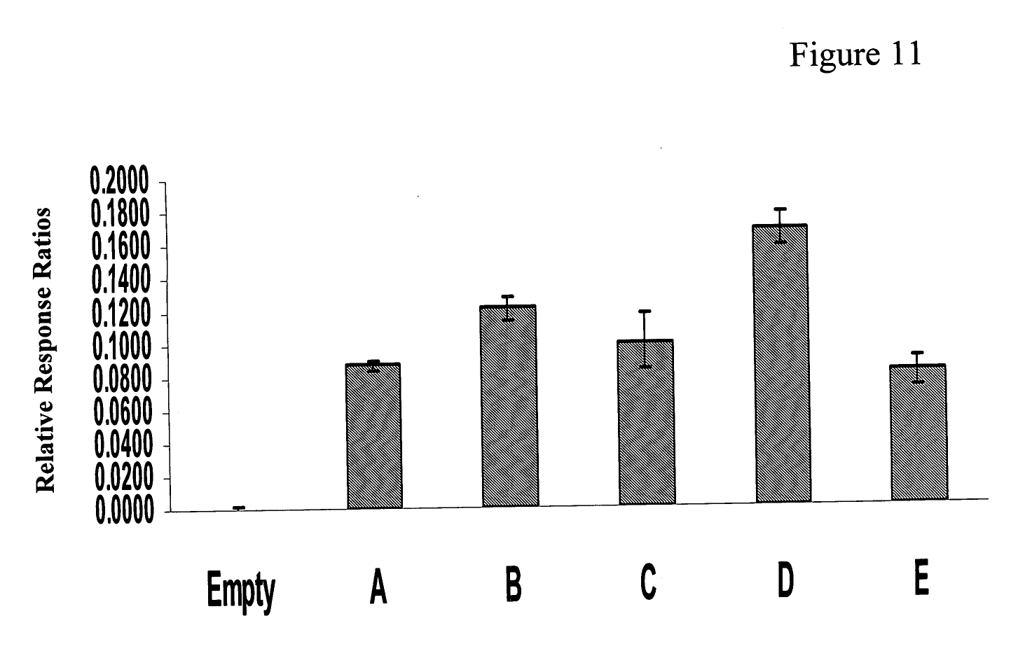

According to alternate preferred embodiments of the invention the quantitative analysis of an RTVP expression level in a biological sample taken from the subject employs an antibody specific to at least a portion of an RTVP-1 protein. Thus, the quantitative assay might be, for example, a western blot (see FIG. 8e), ELISA (enzyme linked immune sorbtion assay), immunohistochemistry or RIA (Radio immunoassay).

The term “antibody” as used in this specification and the accompanying claims includes intact molecules as well as functional fragments thereof, such as Fab, F(ab′)2, and Fv that are capable of binding to macrophages. These functional antibody fragments are defined as follows:
(1) Fab, the fragment which contains a monovalent antigen-binding fragment of an antibody molecule, can be produced by digestion of whole antibody with the enzyme papain to yield an intact light chain and a portion of one heavy chain;
(2) Fab′, the fragment of an antibody molecule that can be obtained by treating whole antibody with pepsin, followed by reduction, to yield an intact light chain and a portion of the heavy chain; two Fab′ fragments are obtained per antibody molecule;
(3) (Fab′)2, the fragment of the antibody that can be obtained by treating whole antibody with the enzyme pepsin without subsequent reduction; F(ab′)2 is a dimer of two Fab′ fragments held together by two disulfide bonds;
(4) Fv, defined as a genetically engineered fragment containing the variable region of the light chain and the variable region of the heavy chain expressed as two chains; and
(5) Single chain antibody (“SCA”), a genetically engineered molecule containing the variable region of the light chain and the variable region of the heavy chain, linked by a suitable polypeptide linker as a genetically fused single chain molecule.
Methods of making these fragments are known in the art. (See for example, Harlow and Lane, Antibodies: A Laboratory Manual, Cold Spring Harbor Laboratory. New York, 1988. incorporated herein by reference).

As used in this specification, the term “epitope” means any antigenic determinant on an antigen to which the paratope of ail antibody binds. Epitopic determinants usually consist of chemically active surface groupings of molecules such as amino acids or carbohydrate side chains and usually have specific three dimensional structural characteristics, as well as specific charge characteristics.

Antibody fragments according to the present invention can be prepared by proteolytic hydrolysis of the antibody or by expression in E. coli or mammalian cells (e.g. Chinese hamster ovary cell culture or other protein expression systems) of DNA encoding the fragment.

Antibody fragments can be obtained by pepsin or papain digestion of whole antibodies by conventional methods. For example, antibody fragments can be produced by enzymatic cleavage of antibodies with pepsin to provide a 5S fragment denoted F(ab′)2. This fragment can be further cleaved using a thiol reducing agent, and optionally a blocking group for the sulfhydryl groups resulting from cleavage of disulfide linkages, to produce 3.5S Fab′ monovalent fragments. Alternatively, an enzymatic cleavage using pepsin produces two monovalent Fab′ fragments and an Fc fragment directly. These methods are described, for example, by Goldenberg, U.S. Pat. Nos. 4,036,945 and 4,331,647, and references contained therein, which patents are hereby incorporated by reference in their entirety. See also Porter, R. R., Biochem. J., 73: 119-126, 1959. Other methods of cleaving antibodies, such as separation of heavy chains to form monovalent light-heavy chain fragments, further cleavage of fragments, or other enzymatic, chemical, or genetic techniques may also be used, so long as the fragments bind to the antigen that is recognized by the intact antibody.

Fv fragments comprise an association of VH and VL chains. This association may be noncovalent, as described in Inbar et al., Proc. Nat'l Acad. Sci. USA 69:2659-62, 1972. Alternatively, the variable chains can be linked by an intermolecular disulfide bond or cross-linked by chemicals such as glutaraldehyde. Preferably, the Fv fragments comprise VH and VL, chains connected by a peptide linker. These single-chain antigen binding proteins (sFv) are prepared by constructing a structural gene comprising DNA sequences encoding the VH and VI, domains connected by an oligonucleotide. The structural gene is inserted into an expression vector, which is subsequently introduced into a host cell such as E. coli. The recombinant host cells synthesize a single polypeptide chain with a linker peptide bridging the two V domains. Methods for producing sFvs are described, for example, by Whitlow and Filpula, Methods, 2: 97-105, 1991; Bird et al, Science 242:423-426, 1988; Packet al., Bio/Technology 11: 1271-77, 1993; and Ladner et al., U.S. Pat. No. 4,946,778, which is hereby incorporated by reference in its entirety.

Another form of an antibody fragment is a peptide coding for a single complementarity-determining region (CDR). CDR peptides (“minimal recognition units”) can be obtained by constructing genes encoding the CDR of an antibody of interest. Such genes are prepared, for example, by using the polymerase chain reaction to synthesize the variable region from RNA of antibody-producing cells. See, for example, Larrick and Fry, Methods, 2: 106-10, 1991.

Humanized forms of non-human (e.g., murine) antibodies are chimeric molecules of immunoglobulins, immunoglobulin chains or fragments thereof (such as Fv, Fab, Fab′, F (ab′)2 or other antigen-binding subsequences of antibodies) which contain minimal sequence derived from nonhuman immunoglobulin. Humanized antibodies include human immunoglobulins (recipient antibody) in which residues form a complementary determining region (CDR) of the recipient are replaced by residues from a CDR of a non-human species (donor antibody) such as mouse, rat or rabbit having the desired specificity, affinity and capacity. In some instances, Fv framework residues of the human immunoglobulin are replaced by corresponding non-human residues. Humanized antibodies may also comprise residues which are found neither in the recipient antibody nor in the imported CDR or framework sequences. In general, the humanized antibody will comprise substantially all of at least one, and typically two, variable domains, in which all or substantially all of the CDR regions correspond to those of a nonhuman immunoglobulin and all or substantially all of the FR regions are those of a human immunoglobulin consensus sequence. The humanized antibody optimally also will comprise at least a portion of an immunoglobulin constant region (Fc), typically that of a human immunoglobulin [Jones et al., Nature, 321:522-525 (1986); Riechmann et al, Nature, 332:323329 (1988); and Presta, Curr. Op. Struct. Biol., 2:593-596 (1992)].

Methods for humanizing non-human antibodies are well known in the art. Generally, a humanized antibody has one or more amino acid residues introduced into it from a source which is non-human. These non-human amino acid residues are often referred to as import residues, which are typically taken from an import variable domain. Humanization can be essentially performed following the method of Winter and co-workers [Jones et al., Nature, 321:522-525 (1986); Riechmann et al., Nature 332:323-327 (1988); Verhoeyen et al., Science, 239:1534-1536 (1988)), by substituting rodent CDRs or CDR sequences for the corresponding sequences of a human antibody. Accordingly, such humanized antibodies are chimeric antibodies (U.S. Pat. No. 4,816.567), wherein substantially less than an intact human variable domain has been substituted by tie corresponding sequence from a non-human species. In practice, humanized antibodies are typically human antibodies in which some CDR residues and possibly some FR residues are substituted by residues from analogous sites in rodent antibodies.

Human antibodies can also be produced using various techniques known in the art, including phage display libraries [Hoogenboom and Winter, J. Mol. Biol., 227:381 (1991); Marks et al., J. Mol. Biol. 222:581 (1991)]. The techniques of Cole et al. and Boerner et al. are also available for the preparation of human monoclonal antibodies (Cole et al., Monoclonal Antibodies and Cancer Therapy, Alan R. Liss, p 77 (1985) and Boerner et al., J. Immunol., 147(1): 86-95 (1991)). Similarly, human antibodies can be made by introducing of human immunoglobulin loci into transgenic animals, e.g., mice in which the endogenous immunoglobulin genes have been partially or completely inactivated. Upon challenge, human antibody production is observed, which closely resembles that seen in humans in all respects, including gene rearrangement, assembly, and antibody repertoire. This approach is described, for example, in U.S. Pat. Nos. 5,545,807; 5.545,806; 5,569,825; 5,625,126; 5,633,425; 5.661,016, and in the following scientific publications: Marks et al., Bio/Technology 10,779-783 (1992); Lonberg et al., Nature 368 856-859 (1994); Morrison, Nature 368 812-13 (1994); Fishwild et al., Nature Biotechnology 14, 845-51 (1996); Neuberger, Nature Biotechnology 14, 826 (1996); Lonberg and Huszar, Intern. Rev. Immunol. 13 65-93 (1995).

The invention is further embodied by a method of influencing a clinical progression of glioma in a subject. The method includes reducing a biological activity of RTVP-1 in the subject.

Practice of this method of treatment is preferably accomplished by administration of a pharmaceutical composition for treating glioma. The pharmaceutical composition further embodies the invention. The pharmaceutical composition includes as an active ingredient a physiologically effective amount of an agent which reduces a biological activity of RTVP-1 in a subject treated with the pharmaceutical composition and a physiologically acceptable carrier and excipient.

Thus, the present invention is further embodied by a method of formulation of a pharmaceutical composition for treatment of glioma. The method includes combining an agent which reduces a biological activity of RTVP-1 with a physiologically acceptable carrier and excipient.

As used herein a “pharmaceutical composition” refers to a preparation of one or more of the active ingredients described herein with other chemical components such as physiologically suitable carriers and excipients. The purpose of a pharmaceutical composition is to facilitate administration of a compound to an organism.

Herein the term “active ingredient” refers to the nucleic acids and/or antibodies accountable for the biological effect.

Hereinafter, the phrases “physiologically acceptable carrier” and “pharmaceutically acceptable carrier” which may be interchangeably used refer to a carrier or a diluent that does not cause significant irritation to an organism and does not abrogate the biological activity and properties of the administered compound. An adjuvant is included under these phases.

Herein the term “excipient” refers to an inert substance added to a pharmaceutical composition to further facilitate administration of an active ingredient. Examples, without limitation, of excipients include calcium carbonate, calcium phosphate, various sugars and types of starch, cellulose derivatives, gelatin, vegetable oils and polyethylene glycols.

Techniques for formulation and administration of drugs may be found in “Remington's Pharmaceutical Sciences,” Mack Publishing Co., Easton, Pa., latest edition, which is incorporated herein by reference.

Suitable routes of administration may, for example, include oral, rectal, transmucosal, especially transnasal, intestinal or parenteral delivery, including intramuscular, subcutaneous and intramedullary injections as well as intrathecal, direct intraventicular, intravenous, intraperitoneal, intranasal, or intraocular injections.

Alternately, one may administer the pharmaceutical composition in a local rather than systemic manner, for example, via injection of the pharmaceutical composition directly into a tissue region of a patient.

Pharmaceutical compositions of the present invention may be manufactured by processes well known in the art, e.g., by means of conventional mixing, dissolving, granulating, dragee-making, levigating, emulsifying, encapsulating, entrapping or lyophilizing processes.

Pharmaceutical compositions for use in accordance with the present invention thus may be formulated in conventional manner using one or more physiologically acceptable carriers comprising excipients and auxiliaries, which facilitate processing of the active ingredients into preparations which can be used pharmaceutically. Proper formulation is dependent upon the route of administration chosen.

For injection, the active ingredients of the pharmaceutical composition may be formulated in aqueous solutions, preferably in physiologically compatible buffers such as Hank's solution, Ringer's solution, or physiological salt buffer. For transmucosal administration, penetrants appropriate to the barrier to be permeated are used in the formulation. Such penetrants are generally known in the art.

For oral administration, the pharmaceutical composition can be formulated readily by combining the active compounds with pharmaceutically acceptable carriers well known in the art. Such carriers enable the pharmaceutical composition to be formulated as tablets, pills, dragees, capsules, liquids, gels, syrups, slurries, suspensions, and the like, for oral ingestion by a patient. Pharmacological preparations for oral use can be made using a solid excipient, optionally grinding the resulting mixture, and processing the mixture of granules, after adding suitable auxiliaries if desired, to obtain tablets or dragee cores. Suitable excipients are, in particular, fillers such as sugars, including lactose, sucrose, mannitol, or sorbitol; cellulose preparations such as, for example, maize starch, wheat starch, rice starch potato starch, gelatin, gum tragacanth, methyl cellulose, hydroxypropylmethylcellulose, sodium carbomethylcellulose; and/or physiologically acceptable polymers such as polyvinylpyrrolidone (PVP). If desired, disintegrating agents may be added, such as cross-linked polyvinyl pyrrolidone, agar, or alginic acid or a salt thereof such as sodium alginate.

Dragee cores are provided with suitable coatings. For this purpose, concentrated sugar solutions may be used which may optionally contain gum arabic, talc, polyvinyl pyrrolidone, carbopol gel, polyethylene glycol, titanium dioxide, lacquer solutions and suitable organic solvents or solvent mixtures. Dyestuffs or pigments may be added to the tablets or dragee coatings for identification or to characterize different combinations of active compound doses.

Pharmaceutical compositions which can be used orally, include push-fit capsules made of gelatin as well as soft, sealed capsules made of gelatin and a plasticizer, such as glycerol or sorbitol. The push-fit capsules may contain the active ingredients in admixture with filler such as lactose, binders such as starches, lubricants such as talc or magnesium stearate and, optionally, stabilizers. In soft capsules, the active ingredients may be dissolved or suspended in suitable liquids, such as fatty oils, liquid paraffin, or liquid polyethylene glycols In addition, stabilizers may be added. All formulations for oral administration should be in dosages suitable for the chosen route of administration.

For buccal administration, the compositions may take the form of tablets or lozenges formulated in conventional manner.

For administration by nasal inhalation, the active ingredients for use according to the present invention are conveniently delivered in the form of an aerosol spray presentation from a pressurized pack or a nebulizer with the use of a suitable propellant, e.g., dichlorodifluoromethane, trichlorofluoromethane, dichloro-tetrafluoroethane or carbon dioxide. In the case of a pressurized aerosol, the dosage unit may be determined by providing a valve to deliver a metered amount Capsules and cartridges of, e.g., gelatin for use in a dispenser may be formulated containing a powder mix of the compound and a suitable powder base such as lactose or starch.

The pharmaceutical composition described herein may be formulated for parenteral administration, e.g., by bolus injection or continuous infusion. Formulations for injection may be presented in unit dosage form, e.g., in ampoules or in multidose containers with optionally, an added preservative. The compositions may be suspensions, solutions or emulsions in oily or aqueous vehicles, and may contain formulatory agents such as suspending, stabilizing and/or dispersing agents.

Pharmaceutical compositions for parenteral administration include aqueous solutions of the active preparation in water-soluble form. Additionally, suspensions of the active ingredients may be prepared as appropriate oily or water based injection suspensions. Suitable lipophilic solvents or vehicles include fatty oils such as sesame oil, or synthetic fatty acids esters such as ethyl oleate, triglycerides or liposomes. Aqueous injection suspensions may contain substances, which increase the viscosity of the suspension, such as sodium carboxymethyl cellulose, sorbitol or dextran. Optionally, the suspension may also contain suitable stabilizers or agents which increase the solubility of the active ingredients to allow for the preparation of highly concentrated solutions.

Alternatively, the active ingredient may be in powder form for constitution with a suitable vehicle, e.g., sterile, pyrogen-free water based solution, before use.

The pharmaceutical composition of the present invention may also be formulated in rectal compositions such as suppositories or retention enemas, using, e.g., conventional suppository bases such as cocoa butter or other glycerides.

Pharmaceutical compositions suitable for use in context of the present invention include compositions wherein the active ingredients are contained in an amount effective to achieve the intended purpose. More specifically, a therapeutically effective amount means an amount of active ingredients (nucleic acid construct) effective to prevent, alleviate or ameliorate symptoms of a disorder (e.g., ischemia) or prolong the survival of the subject being treated.

Determination of a therapeutically effective amount is well within the capability of those skilled in the art, especially in light of the detailed disclosure provided herein.

For any preparation used in the methods of the invention, the therapeutically effective amount or dose can be estimated initially from in vitro and cell culture assays. For example, a dose can be formulated in animal models to achieve a desired concentration or titer. Such information can be used to more accurately determine useful doses in humans.

Toxicity and therapeutic efficacy of the active ingredients described herein can be determined by standard pharmaceutical procedures in vitro, in cell cultures or experimental animals. The data obtained from these in vitro and cell culture assays and animal studies can be used in formulating a range of dosage for use in humans. The dosage may vary depending upon the dosage form employed and the route of administration utilized. The exact formulation, route of administration and dosage can be chosen by the individual physician in view of the patient's condition. (See e.g., Fingl, et al., 1975, in “The Pharmacological Basis of Therapeutics”, Ch. 1 p. 1).

Dosage amount and interval may be adjusted individually to provide plasma or brain levels of the active ingredient that are sufficient to induce or suppress angiogenesis (minimal effective concentration, MEC). The MEC will vary for each preparation, but can be estimated from in vitro data. Dosages necessary to achieve the MEC will depend on individual characteristics and route of administration. Detection assays can be used to determine plasma concentrations.

Depending on the severity and responsiveness of the condition to be treated, dosing can be of a single or a plurality of administrations, with course of treatment lasting from several days to several weeks or until cure is effected or diminution of the disease state is achieved.

The amount of a composition to be administered will, of course, be dependent on the subject being treated, the severity of the affliction, the manner of administration, the judgment of the prescribing physician, etc.

Compositions of the present invention may, if desired, be presented in a pack or dispenser device, such as an FDA approved kit, which may contain one or more unit dosage forms containing the active ingredient. The pack may, for example, comprise metal or plastic foil, such as a blister pack. The pack or dispenser device may be accompanied by instructions for administration. The pack or dispenser may also be accommodated by a notice associated with the container in a form prescribed by a governmental agency regulating the manufacture, use or sale of pharmaceuticals, which notice is reflective of approval by the agency of the form of the compositions or human or veterinary administration. Such notice, for example, may be of labeling approved by the U.S. Food and Drug Administration for prescription drugs or of an approved product insert. Compositions comprising a preparation of the invention formulated in a compatible pharmaceutical carrier may also be prepared, placed in an appropriate container, and labeled for treatment of an indicated condition, as if further detailed above.

A reduction of the biological activity of RTVP-1 by a pharmaceutical composition may be accomplished in a variety of ways. Each of these ways is a preferred embodiment of the invention. By way of non-limiting example, reduction of biological activity may be accomplished by reducing a level of transcription of an RTVP-1 gene, decreasing a stability of an RTVP-1 mRNA transcript, decreasing a level of translation of an RTVP-1 mRNA transcript, reducing a level of activity of an RTVP-1 protein or reducing the stability of an RTVP-1 protein.

Reducing a level of transcription of an RTVP-1 gene may be accomplished by, for example, use of antisense RNA.

Historically, use of antisense oligonucleotides to specifically block transcription of designated mRNA's has required solution of two problems. The first problem is delivery of the oligonucleotide into the cytoplasm of the appropriate cells, and the second problem is design of an oligonucleotide which binds the designated mRNA in a way which inhibits translation. In the course of time, a number of delivery strategies have been developed (e.g. Luft (1998) J Mol Med 76(2):75-6; Kronenwett et al. (1998) Blood 91(3):852-62; Raju et al. (1997) Bioconjug Chem 8(6):935-40; Lavigne et al. (1997) Biochem Biophys Res Commun 237(3):566-71 and Aoki et al. (1997) Biochem Biophys Res Commun 231(3):540-5). Subsequently, a means of predicting efficiency of specific oligonucleotides using an in vitro system was published (Matveeva et al. (1998) Nature Biotechnology 16, 1374-1375). A year later preclinical studies provided evidence of antisenise effects in vitro and in vivo, and phase 1 clinical trials demonstrated safety, feasibility and activity of antisense oligonucleotides for the treatment of cancer and commercial development was being undertaken (Holmund et al. (1 999) Curr Opin Mol Ther 1 (3):372-85). An independent review that same year reported that treatment of hematological malignancies via antisense oligonucleotides targeting c-myb gene, p53 and Bcl-2 had entered clinical trials and had been shown to be tolerated by patients (Gerwitz (1999) CUff Opin Mol Ther 1 (3):297-306). More recently, antisense-mediated suppression of human heparanase gene expression has been reported to inhibit pleural dissemination of human cancer cells in a mouse model (Uno et al. (2001) Cancer Res 61(21):7855-60). These articles indicate that issues of delivery have essentially been addressed. At the same time, development of an algorithm for identifying those sequences with the highest predicted binding affinity for their target mRNA based on a thermodynamic cycle that accounts for the energetics of structural alterations in both the target mRNA and the oligonucleotide had been developed (Walton et al. (1999) Biotechnol Bioeng 65(1):1-9). This algorithm successfully designed antisense oligonucleotides for rabbit beta-globin (RBG) and mouse tumor necrosis factor-alpha (TNFalpha) transcripts. The same research group has more recently reported that the antisense activity of rationally selected oligonucleotides against three model target mRNAs (human lactate dehydrogenase A and B and rat gp130) in cell culture as evaluated by a kinetic PCR technique proved effective in almost all cases, including tests against three different targets in two cell types with phosphodiester and phosphorothioate oligonucleotide chemistries. In light of this algorithm for designing antisense oligonucleotides and the wide variety of delivery systems reported in the literature, it seems that reduction of translation from transcripts having a known sequence by antisense oligonucleotides has become a matter of routine calibration.

For example Jayarman et al. (Biochim Biophys Acta (2001) 1520(2):105-14) teach methods for rational selection and quantitative evaluation of antisense oligonucleotides using a prediction algorithm for identifying high affinity antisense oligonucleotides based on mRNA oligonucleotide hybridization.

Stability of an RTVP-1 mRNA may be decreased, for example, by use of ribozymes with specificity to at least a portion of the RTVP-1 mRNA transcript.

Thus, the pharmaceutical composition of the present invention may include, as an active ingredient a ribozyme comprising an antisense oligonucleotide specific to a desired portion of an RTVP-1 transcript and a ribozyme sequence fused thereto. Such a ribozyme is readily synthesizable using solid phase oligonucleotide synthesis.

Ribozymes are being increasingly used for the sequence-specific inhibition of gene expression by the cleavage of mRNAs encoding proteins of interest [Welch et al., “Expression of ribozymes in gene transfer systems to modulate target RNA levels.” Curr Opin Biotechnol. Oct. 9, 1998;(5):486-96]. The possibility of designing ribozymes to cleave any specific target RNA has rendered them valuable tools in both basic research and therapeutic applications. In the therapeutics area, ribozymes have been exploited to target viral RNAs in infectious diseases, dominant oncogenes in cancers and specific somatic mutations in genetic disorders [Welch et al., “Ribozyme gene therapy for hepatitis C virus infection.” Clin Diagn Virol. 1998 Jul. 15; 10(2.-3):163-71.]. Most notably, several ribozyme gene therapy protocols for HIV patients are already in Phase I trials. More recently, ribozymes have been used for transgenic animal research, gene target validation and pathway elucidation. Several ribozymies are in various stages of clinical trials. ANGIOZYME was the first chemically synthesized ribozyme to be studied in human clinical trials. ANGIOZYME specifically inhibits formation of the VEGF-r (Vascular Endothelial Growth Factor receptor), a key component in the angiogenesis pathway. Ribozyme Pharmaceuticals, Inc., as well as other firms have demonstrated the importance of anti-angiogenesis therapeutics in animal models. HEPTAZYME, a ribozyme designed to selectively destroy Hepatitis C Virus (HCV) RNA, was found effective in decreasing Hepatitis C viral RNA in cell culture assays (Ribozyme Pharmaceuticals, Incorporated—WEB home page).

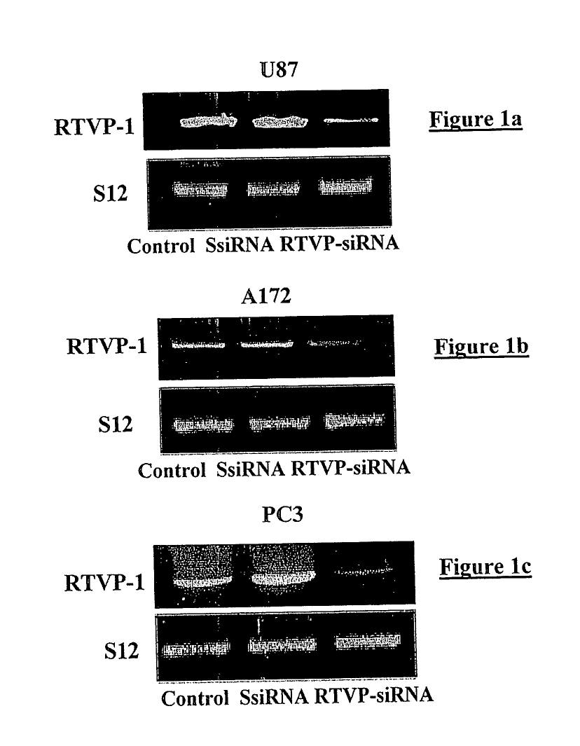

A most preferred embodiment of the present invention relies upon influencing stability of an RTVP-1 mRNA transcript by administration of siRNA to the subject. SiRNAs destabilize transcripts by facilitating enzymatic cleavage thereof. Non-limiting examples of siRNA suited for use in this context are pairs of complementary RNA sequences such as SEQ. ID. NOs: 1 and 2; SEQ. ID. NOs: 3 and 4; SEQ. ID. NOs: 31 and 32; SEQ. ID. NOs: 26 and 27; and SEQ. ID. NOs: 28 and 29. SEQ. ID. NOs.: 26; 27; 28 and 29 produce RNA with hairpin structures. These hairpin structures are believed to increase stabilitity of the siRNA and are often referred to as shRNA. Usefulness of these siRNAs is demonstrated in FIGS. 1a, 1b, 1c, 2b, 2c, 3a, 3b , 3c and explained in example 3 hereinbelow.

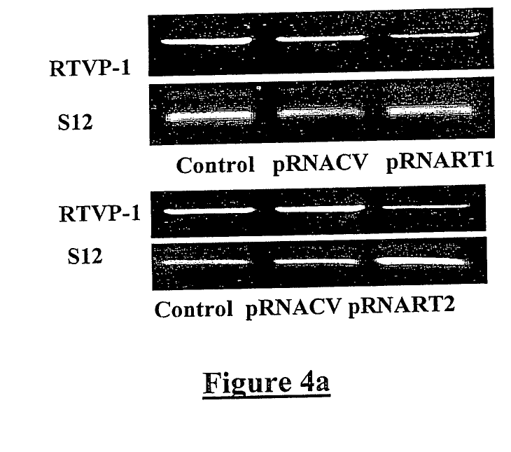

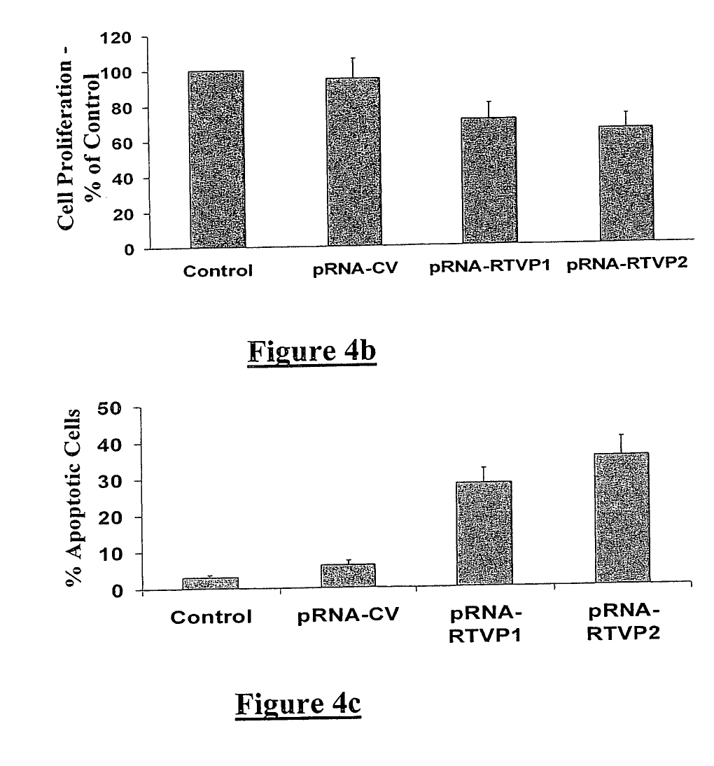

Sustained delivery of siRNAs by incorporation into a plasmid vector is demonstrated in FIGS. 4a, 4b and 4c and explained in example 3. This experiment serves as a demonstration that a wide variety of gene therapy approaches have potential utility in the context of siRNA delivery for purposes of glioma treatment.

The ability to efficiently and stably produce and deliver sufficient amounts of siRNA to cells in vitro provide the basis for the development of siRNA as gene-specific therapeutic agents for the treatment of various disorders including cancer. Indeed, initial in-vivo studies reported effective gene suppression in mice by siRNAs. In addition, recent studies demonstrated the use of plasmid and viral vectors for the transcription of short-hairpin RNAs, both in vitro and in vivo. Using these expression systems, gene expression was stably inhibited (Wall and Shi, Lancet 2003; Sioud, Trends Phannacol Sci, 2004).

Gene therapy as used herein refers to the transfer of genetic material (e.g. DNA or RNA) of interest into a host to treat glioma, retard glioma progression or ameliorate glioma symptoms. The genetic material of interest encodes a siRNA product whose production in vivo is desired. For review see, in general, the text “Gene Therapy” (Advances in Pharmacology 40, Academic Press, 1997).

Two basic approaches to gene therapy have evolved: (1) ex vivo and (2) in vivo gene therapy. In ex vivo gene therapy cells are removed from a subject, and while being cultured are treated in vitro. A vector designed to deliver the desired product (siRNA in this case) is introduced into the cell via an appropriate gene delivery vehicle/method (transfection, transduction, homologous recombination, etc.) and an expression system as needed and then the modified cells are expanded in culture and returned to the subject. These modified re-implanted cells have been shown to express the transfected genetic material in situ.

In in vivo gene therapy, target cells are not removed from the subject rather the genetic material to be transferred is introduced into the cells of the subject organism in situ, that is within the subject. These genetically altered cells have been shown to express the transfected genetic material in situ.

The gene expression vehicle is capable of delivery/transfer of heterologous nucleic acid into a host cell. The expression vehicle may include elements to control targeting, expression and transcription of the nucleic acid in a cell selective manner as is known in the art.

The expression vehicle may include, for example a promoter for controlling transcription of the heterologous material (e.g. siRNA) which may be either a constitutive, tissue specific (e.g. promoter fragments A, B, C, D and E as described hereinbelow; SEQ. ID. NOs.: 21-25 respectively), cell type specific or inducible promoter to allow selective transcription. Enhancers that may be required to obtain necessary transcription levels can optionally be included. Enhancers are generally any nontranslated DNA sequence which works contiguously with the coding sequence (in cis) to change the basal transcription level dictated by the promoter. The expression vehicle can also include a selection gene as described herein below.

Vectors can be introduced into cells or tissues by anyone of a variety of known methods within the art. Such methods can be found generally described in Sambrook et al, Molecular Cloning: A Laboratory Manual, Cold Springs Harbor Laboratory, New York 1989, 1992), in Ausubel et al., Current Protocols in Molecular Biology, John Wiley and Sons, Baltimore, Md. 1989), Chang et al., Somatic Gene Therapy, CRC Press, Ann Arbor, Mich. 1995), Vega et al., Gene Targeting, CRC Press, Ann Arbor Mich. (995), Vectors: A Survey of Molecular Cloning Vectors and Their Uses, Butterworths, Boston Mass. 1988) and Gilboa et al. (Biotechniques 4 (6): 504-512, 1986) and include, for example, stable or transient transfection, lipofection, electroporation and infection with recombinant viral vectors. In addition, see U.S. Pat. No. 4,866,042 for vectors involving the central nervous system and also U.S. Pat. Nos. 5,464,764 and 5,487,992 for positive-negative selection methods.

Introduction of nucleic acids by infection offers several advantages over the other listed methods. Higher efficiency can be obtained due to their infectious nature. Moreover, viruses are very specialized and typically infect and propagate in specific cell types. Thus, their natural specificity can be used to target the vectors to specific cell types in vivo or within a tissue or mixed culture of cells. Viral vectors can also be modified with specific receptors or ligands to alter target specificity through receptor mediated events.

A specific example of DNA viral vector introducing and expressing recombination sequences is the adenovirus-derived vector Adenop53TK. This vector expresses a herpes virus thymidine kinase (TK) gene for either positive or negative selection and an expression cassette for desired recombinant sequences. This vector can be used to infect cells that have an adenovirus receptor which includes most cancers of epithelial origin as well as others. This vector as well as others that exhibit similar desired functions can be used to treat a mixed population of cells and can include, for example, an in vitro or ex vivo culture of cells, a tissue or a human subject.

Features that limit expression to particular cell types can also be included. Such features include, for example, promoter and regulatory elements that are specific for the desired cell type.

In addition, recombinant viral vectors are useful for in vivo expression of a desired nucleic acid because they offer advantages such as lateral infection and targeting specificity. Lateral infection is inherent in the life cycle of, for example, retrovirus and is the process by which a single infected cell produces many progeny virions that bud off and infect neighboring cells. The result is that a large area becomes rapidly infected, most of which was not initially infected by the original viral particles. This is in contrast to vertical-type of infection in which the infectious agent spreads only through daughter progeny Viral vectors can also be produced that are unable to spread laterally. This characteristic can be useful if the desired purpose is to introduce a specified gene into only a localized number of targeted cells.

As described above, viruses are very specialized infectious agents that have evolved, in many cases, to elude host defense mechanisms. Typically, viruses infect and propagate in specific cell types. The targeting specificity of viral vectors utilizes its natural specificity to specifically target predetermined cell types and thereby introduce a recombinant gene into the infected cell. The vector to be used in the methods of the invention will depend on desired cell type to be targeted and will be known to those skilled in the art. For example, if breast cancer is to be treated then a vector specific for such epithelial cells would be used. Likewise, if diseases or pathological conditions of the hematopoietic system are to be treated, then a viral vector that is specific for blood cells and their precursors, preferably for the specific type of hematopoietic cell, would be used.

Retroviral vectors can be constructed to function either as infectious particles or to undergo only a single initial round of infection. In the former case, the genome of the virus is modified so that it maintains all the necessary genes, regulatory sequences and packaging signals to synthesize new viral proteins and RNA. Once these molecules are synthesized, the host cell packages the RNA into new viral particles which are capable of undergoing further rounds of infection. The vector's genome is also engineered to encode and express the desired recombinant gene. In the case of non-infectious viral vectors, the vector genome is usually mutated to destroy the viral packaging signal that is required to encapsulate the RNA into viral particles. Without such a signal, any particles that are formed will not contain a genome and therefore cannot proceed through subsequent rounds of infection. The specific type of vector will depend upon the intended application. The actual vectors are also known and readily available within the art or can be constructed by one skilled in the art using well-known methodology.

The recombinant vector can be administered in several ways. If viral vectors are used, for example, the procedure can take advantage of their target specificity and consequently, do not have to be administered locally at the diseased site. However, local administration can provide a quicker and more effective treatment. Administration can also be performed by, for example, intravenous or subcutaneous injection into the subject. Injection of the viral vectors into a spinal fluid can also be used as a mode of administration, especially in the case of neuro-degenerative diseases. Following injection, the viral vectors will circulate until they recognize host cells with appropriate target specificity for infection.

Alternately or additionally, the active ingredient of the pharmaceutical composition may employ an antibody specific to at least a portion of an RTVP-1 protein to reduce biological activity of RTVP-1.

The present invention is additionally embodied by an isolated nucleic acid sequence characterized by an ability to positively regulate a downstream gene. The sequence includes at least a functional portion of SEQ. ID. NO.: 25.

Examples of functional portions of SEQ. ID. NO.: 25 include, but are not limited to SEQ. ID. NOs.: 21; 22; 23 and 24. The alignment of these fragments is depicted in FIG. 9 and their relative promoter activity with respect to a downstream reporter gene is demonstrated in FIGS. 10a and 10b. Example 4 explains these figures. SEQ. ID. NO. 25 and active portions thereof are typically each refereed to as “a promoter” and that term is employed hereinbelow.

Such a promoter may be used to specifically deliver therapeutic agents to glioma cells as illustrated in Example 5 hereinbelow.

The scope of the invention includes an expression vector including a promoter as detailed hereinabove. The phrase “expression vector” as used in this specification and the accompanying claims specifically includes, but is not limited to, viral vectors (e.g. adenovirus or lentivirus vectors), plasmids, phage vectors, pagemids, cosmids and other vectors which are known or will become known to those familiar with recombinant nucleic acid technology. The scope of the invention further includes a mammalian cell transfected with such an expression vector. The scope of the invention further includes an animal into which such cells have been introduced.

The scope of the invention further includes a transgenic animal including at least one exogenous copy of the promoter within its genome.

The present invention has the potential to provide transgenic gene and polymorphic gene animal and cellular (cell lines) models as well as for knockout models. The term “knockout”, as used herein, refers to a reduction of RTVP-1 activity to zero, by any of the various methods detailed hereinabove. These models may be constructed using standard methods known in the art and as set forth in U.S. Pat. Nos. 5,487,992, 5,464,764, 5,387,742, 5,360,735, 5,347,075, 5.298,422, 5,288,846, 5,221,778, 5,175,385, 5,175,384, 5,175,383, 4,736,866 as well as Burke and Olson, Methods in Enzymology, 194:251-270 1991); Capecchi, Science 244:1288-1292 1989); Davies et al., Nucleic Acids Research, 20 (II) 2693-2698 1992); Dickinson et al., Human Molecular Genetics, 2(8): 1299-1302 1993); Duff and Lincoln. “Insertion of a pathogenic mutation into a yeast artificial chromosome containing the human APP gene and expression in ES cells”, Research Advances in Alzheimer's Disease and Related Disorders, 1995; Huxley et al., Genomics, 9:742-750 1991); Jakobovits et al., Nature, 362:255-2611993); Lamb et al., Nature Genetics, 5: 22-29 1993); Pearson and Choi, Proc. Natl. Acad. Sci. USA 1993). 90: 10578-82; Rothstein, Methods in Enzymology, 194:281-3011991); Schedl et al., Nature, 362: 258-2611993); Strauss et al., Science, 259: 1904-1907 1993). Further, patent applications WO 94/23049, WO93/14200, WO 94/06908. WO 94/28123 also provide information.

All such transgenic gene and polymorphic gene animal and cellular (cell lines) models and knockout models for cell proliferation disorder, including but not limited to glioma, or therapy thereof, derived from claimed embodiments of the present invention, constitute preferred embodiments of the present invention. At least two types of animal models are envisioned.

In the first type, a construct containing an RTVP-1 promoter as detailed hereinabove and hereinbelow drives expression of a downstream tranforming gene (e.g. T antigen) is used to either transform neuronal cells, or to generate transgenic mice. This provides an animal model for glioma. Transgenic mice produced according to this strategy should spontaneously develop glioma. Alternately, cells transformed with such a construct are implanted into normal mice, for example in the brain, in order to create mice with glioma tumors.

In the second type a construct containing an RTVP-1 promoter as detailed hereinabove and hereinbelow drives expression of a downstream anti-RTVP-1 molecule (e.g. antisense RNA; siRNA; antibody or ribozyme) is used to generate transgenic mice. This provides an animal model for resistance to glioma Transgenic mice produced according to this strategy should show significantly reduced incidence of glioma in the face of exposure to carcinogens.

According to additional preferred embodiments of the invention, delivery of active ingredients may be accomplished using biodegradable polymers in the form of nanoparticles, for example, nanoparticles containing SiRNa, shRNA or antibodies as detailed hereinabove.

It is expected that during the life of this patent many relevant quantitative analytic techniques will be developed and the scope of the “diagnosing” is intended to include all such new technologies a priori.

Additional objects, advantages, and novel features of the present invention will become apparent to one ordinarily skilled in the art upon examination of the following examples, which are not intended to be limiting. Additionally, each of the various embodiments and aspects of the present invention as delineated hereinabove and as claimed in the claims section below finds experimental support in the following examples.

### EXAMPLES

Reference is now made to the following examples, which together with the above descriptions, illustrate the invention in a non limiting fashion.

Generally, the nomenclature used herein and the laboratory procedures utilized in the present invention include molecular, biochemical, microbiological and recombinant DNA techniques. Such techniques are thoroughly explained in the literature. See, for example, “Molecular Cloning: A Laboratory Manual” Sambrook et al., (1989); “Current Protocols in Molecular Biology” Volumes I-III Ausubel, R. M., ed. (J 994); Ausubel et al, “Current Protocols in Molecular Biology”, John Wiley and Sons, Baltimore. Md. (1989); Perbal, “A Practical Guide to Molecular Cloning”, John Wiley & Sons, New York (1988); Watson et al, “Recombinant DNA”, Scientific American Books. New York; Birren et al. (eds) “Genome Analysis: A Laboratory Manual Series”, Vols. 1-4, Cold Spring Harbor Laboratory Press, New York (1998); methodologies as set forth in U.S. Pat. Nos. 4,666,828; 4,68.3,202; 4,801,531; 5,192,659 and 5,272,057; “Cell Biology: A Laboratory Handbook”. Volumes I-III Cellis, J. E., ed. (1994); “Culture of Animal Cells—A Manual of Basic Technique” by Freshney, Wiley-Liss, N.Y. (1994), Third Edition; “Current Protocols in Immunology” Volumes I-III Coligan J. E., ed. (1994); Stites et al. (eds), “Basic and Clinical Immunology” (8th Edition), Appleton & Lange, Norwalk, Conn. (1994); Mishell and Shiigi (eds), “Selected Methods in Cellular Immunology”, W. H. Freeman and Co., New York (1980); available immunoassays are extensively described in the patent and scientific literature, see, for example, U.S. Pat. Nos. 3,791,932; 3,839,153; 3,850,752; 3,850,578; 3,853,987; 3,867,517; 3,879,262; 3,901,654; 3,935,074; 3,984,533; 3,996,345; 4,034,074; 4,098,876; 4,879,219; 5,011,771 and 5,281,521; “Oligonucleotide Synthesis” Gait. M. J., ed. (1984); “Nucleic Acid Hybridization” Hames, B. D., and Higgins S. J., eds. (1985); “Transcription and Translation” Hames, B. D., and Higgins S. J., eds. (1984); “Animal Cell Culture” Freshney, R. I., ed. (1986); “Immobilized Cells and Enzymes” IRL Press, (1986); “A Practical Guide to Molecular Cloning” Perbal, B., (1984) and “Methods in Enzymology” Vol. 1-317, Academic Press; “PCR Protocols: A Guide To Methods And Applications”, Academic Press, San Diego, Calif. (1990); Marshak et al., “Strategies for Protein Purification and Characterization—A Laboratory Course Manual” CSHL Press (1996); all of which are incorporated by reference as if fully set forth herein. Other general references are provided throughout this document. The procedures therein are believed to be well known in the art and are provided for the convenience of the reader. All the information contained therein is incorporated herein by reference.

Before presenting examples, reference is made to the following materials and methods employed in performance of experiments described in the examples.

### Materials and Methods

Tumors Tumors were classified according to the World Health Organization criteria into the various subtypes of low-grade astrocytomas, anaplastic astrocytomas and glioblastoma multiforme. Histopathological diagnoses were made according to the World Health Organization guidelines and evaluated in formalin-fixed paraffin-embedded hematoxylin/eosin-stained tissue slices. Tumors were collected from patients operated on at Hadassah University Hospital or at Henry Ford Hospital, Detroit, Mich. Fresh tissue was frozen immediately following surgery in liquid nitrogen and stored at −70° C. until processing. Sample collection and processing were performed according to the regulations of the committee on research involving human subjects of the Organization Institutional Review Board (IRB).

RNA extraction and RT-PCR: For extraction of RNA, frozen tissues were washed from blood in ice-cold PBS, were homogenized and total RNA was extracted using RNAeasy (Qiagen) according to manufacturer's instructions. RNA was dissolved in 20 μl of DEPC-treated H₂O.

One microgram of total RNA was transcribed into cDNA using the Reverse Transcriptase System (Promega) using pd(N)6 random nucleotides. Relative levels of RTVP-1 mRNA were estimated by a semi-quantitative polymerase chain reaction (PCR) as compared to the mRNA levels of the ribosomal protein S-12. The cDNA products, 1 μg—for PCR with RTVP primers and 0.25 μg—for PCR with S-12 primers, were resuspended in a total volume of 50 μl containing 1 unit of Taq DNA Polymerase (Takara, Japan), 200 μM each of dATP, dCTP, dGTP, dTTP, 1× reaction buffer provided by the manufacturer and 50 pmol of primers.

The following primers were used for semi-quantitative RT-PCR:

For RTVP-1 three sets of primers were used that gave similar results:

(SEQ. ID. No: 7)

Forward primer: 5′-GGAAGGCATTGCTGCTGG

(SEQ. ID. No: 8)

reverse primer: 5′-CCTCAATGACATCCTTGG
558 bp product

(SEQ. ID. No: 9)

Forward primer. 5′-TGAGCACTCAGGCAATCACACTCT

(SEQ. ID. No: 10)

Reverse primer. 5′-AGTGCTGGGTCCCAAGTCATGTAT
271 bp product

(SEQ. ID. No: 11)

Forward primer. 5′-ATGGGCCAGCCTGGATATACAACA

(SEQ. ID. No: 12)

Reverse primer. 5′-CACAAGCTGCACCCAAACTTCACT
422 bp product
For S12:

(SEQ. ID. No: 13)

Forward primer 5′-GGAAGGCATTGCTGCTGG

(SEQ. 10. No: 14)

Reverse primer: 5′-CCTCAATGACATCCTTGG
285 bp product

Primers for S-12 and RTVP-1 span exon-intron junctions in order to avoid amplification of contaminating genomic DNA. Amplification step consisted of 95° C. for 2 min and 27 (for RTVP-1) or 30 (for S-12) cycles of 95° C for 30 s, 65° C. for 30 sec for 90 sec. In a preliminary study, each cDNA was amplified in serial of 25, 30, 35 and 40 cycles to obtain data within the linear-range of the assay. PCR products were size-fractionated by electrophoresis in 2% agarose gels and were stained with ethidiuni bromide. For molecular weight markers, we used 50 bp DNA ladder (MWXIII, Boehringer Mannheim). The specificity of the PCR product was verified by DNA sequencing.

Quantitation of PCR products. Bands from RT-PCR using RTVP-1 and S12 primers were scanned and quantitated by Scion Image. RTVP-1 products were normalized to S12 products to control for differences in loading, sample integrity, etc.

Cell Lines. The following human glioma cell lines from ATCC were used in these studies: U87 (HTB-14), A172 (CRL-1620), U118 (HTB-15), LN-229 (CRL-2611), LN-18 (CRL-2610) and T98G (CRL-1690). The U251 glioma cell line was obtained from Dr. Oliver Bogler, Department of Neurosurgery, Henry Ford Hospital. Detroit, Mich. All these cell lines were grown in medium consisting of Dulbecco's modified Eagle's medium (DMEM) containing 10% heat-inactivated fetal calf serum, 2 mM glutaniine, penicillin (50 U/ml) and streptomycin (0.05 mg/ml). Cells were splitted every four days using 0.25% trypsin. Normal fetal astrocytes were obtained from Cambrex (Walkersville, Md., USA). Cells were grown in an astrocyte specific medium provided by Cambrax.

Cell nucleofection: Cells were transfected by nucleofection using the Nucleofector system from Amaxa GmbH (Cologne, Germany). The Nucleofector technology is a highly efficient non-viral gene transfer method for most primary cells and for hard-to-transfect cell lines. The technology is an improved version of electroporation. Cell-type specific combinations of electrical current and solutions increase the capacity to transfer polyanionic macromolecules directly into the nucleus. Thus, cells with limited potential to divide, like many primary cells, become accessible for efficient gene transfer. Conditions for each cell type were optimized using manufactures' guidelines. After centrifugation, 1×10⁶ cells were suspended in 100 μl pre-warmed Nucleofector Solution Kit V, containing 1 μg of RTVP-promoter-pGL3-plasmid and 0.5 μg of CV-pGL3-plasmid in a 2 mm electroporation cuvette (Amaxa GmbH, Cologne; Germany). The Nucleofector Kits are cell type specific solutions and commercially available for different cell types (Amaxa GmbH). The samples were kept in the cuvette only for the time of the pulse. The cells were transfected with the electrical setting A-23. After nucleofection with optimized programs the cells were transferred immediately into pre-warmed complete DMEM medium and cultured for 48 h in white 96-well microplates.

Luciferase activity assay: After the indicated incubation times, an equal volume of luciferase substrate (Steady Glo reagent, Promega; USA) was added to all samples, and the luminescence was measured in a microplate luminometer (SPECTRAFluor Plus, Tecan, Austria).

siRNA Transfections: siRNA duplexes (siRNAs) were synthesized and purified by Dharmacon (Lafayette, Colo.). The siRNA sequences are described hereinbelow and set forth in the SEQ. ID. NOs: 1 and 2; SEQ. ID. NOs: 3 and 4; SEQ. ID. NOs: 31 and 32; SEQ. ID. NOs: 26 and 27; and SEQ. ID. NOs: 28 and 29. A scrambled sequence (SEQ. ID. NOs.: 5 and 6) was used as a negative control. Transfection of siRNAs was performed using OligoFectamine (Invitrogen, Carlsbad, Calif.) according to the manufacturer's instruction. RTVP-1 mRNA levels were reduced by about 70% after 48 hr of treatment and by 90% after 72 hr of treatment as deteniined by semi-quantitative RT-PCR analysis.

Preparation of Cell Homogenate for immumoblot analysis: Cells were scraped and centrifuged at 1,400 rpm for 10 min. The supernatants were aspirated and the cell pellets were resuspended in 100 μl of lysis buffer (25 mM Tris-HCI, pH 7.4, 50 mM NaCI, 0.5% Na deoxycholate; 2% NP-40; 0.2% SDS; 1 mM PMSF; 50 μg/ml aprotinin; 50 μg leupeptin; 0.5 mM Na₃V0₄) on ice for 15 min. 2× sample buffer was added and the samples were boiled for 5 min.

Immunoblot Analysis: Lysates (40 μg protein) were resolved by SDS-PAGE (10%) and were transferred to nitrocellulose membranes. The membranes were blocked with 5% dry milk in phosphate-buffered saline and subsequently stained with the rabbit anti-RTVP-1 primary antibody. Specific reactive bands were detected using a goat anti-rabbit or goat anti-mouse IgG conjugated to horseradish peroxidase (BioRad, Hercules, Calif.) and the immunoreactive bands were visualized by the ECL, Western blotting detection kit (Amersham, Arlington Heights, Ill.).

Antibody preparation: Using commercially available services (Sigma; Israel), a synthetic peptide: DIENEDFIKDCVRIHNK (SEQ. ID. NO.: 30) was prepared and used to immunize a rabbit. The resultant anti-RTVP-1 antibody was affinity purified against the synthetic peptide and was used in Western blot analysis as described hereinabove.

### Example 1

### Difference in RTVP-1 Expression Between Glioma Cell Lines and Normal Astrocytes

RTVP-1 expression levels were initially examined in a number of glioma cells lines and in normal human astrocytes. Samples of mRNA were subjected to RT-PCR as detailed hereinabove and run on agarose gels containing formaldehyde and ethidium bromide. S12 was employed as a control for loading. As summarized in FIG. 5, all the glioma cell lines examined expressed high levels of RTVP-1 as detected by semi-quantitative RT-PCR. In contrast, very low levels of RTVP-1 were detected in the normal human astrocytes. This result demonstrates upregulation of RTVP-1 in glial transformation.

### Example 2

### Differential Expression of RTVP-1 in Glial Tumors with Different Degree of Malignancy

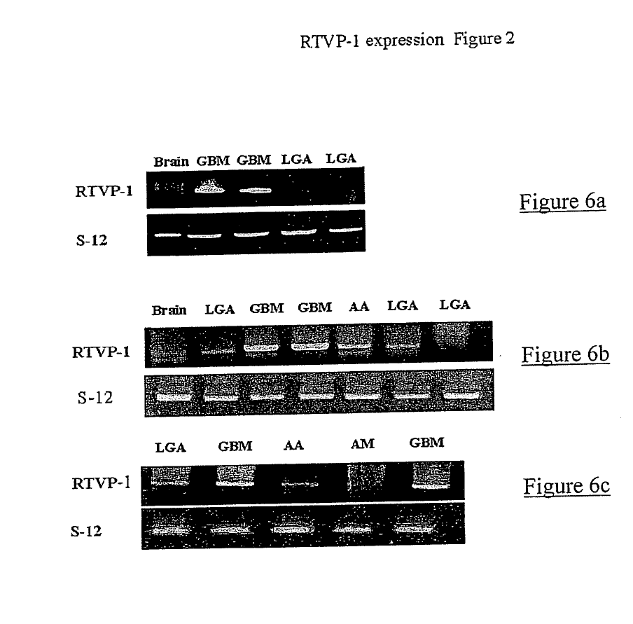

Low-grade gliomas (grade II), anaplastic astrocytomas (grade III) and glioblastoma multiforme (GBM, grade IV) are characterized by a different profile of mutations and genetic alterations. For example, p53 mutations are common in the low-grade tumors, whereas overexpression and mutation in the EGFR are common in the GBM. However, no easily measured molecular marker was previously available. Therefore, quantitative analysis of RTVP-1 expression levels in glioma tumors with different degree of malignancy was undertaken in an attempt to establish the potential clinical prognostic value of RTVP-1 expression level. RT-PCR as described hereinabove was employed for quantitation. Results are presented in FIGS. 6a, 6b and 6c which show the results of the RT-PCR of the different tumors in three separate experiments [adult brains (brain), low-grade astrocytomas (LGA), anaplastic astrocytomas (AA), glioblastoma multiforme (GBM) and anaplastic meningiomas (AM)].

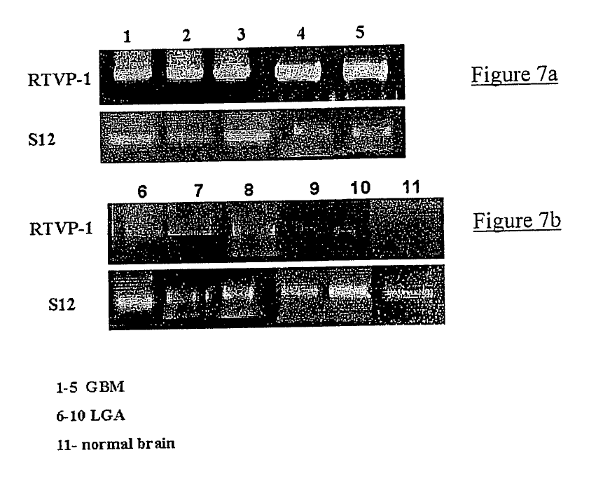

Normal brain cells did not express detectable levels of RTVP-1 mRNA. Similarly, RTVP-1 expression was below the threshold of detection in benign and malignant meningiomas. Low-grade gliomas exhibited RTVP-1 expression levels at approximately the threshold of detection. Anaplastic astrocytomas expressed low, but detectable, levels of RTVP-1. In sharp contrast, all the GBM examined expressed high levels of RTVP-1. These results were consistently reproduced using additional tissue samples as they became available from patients (FIGS. 7a; 7b; 8a; 8b; 8c and 8d).

A polyclonal anti-RTVP-1 antibody as described hereinabove in methods and materials was used for immunoblotting of tissue extracts. Expression of RTVP-1 protein was not detectable in normal brain. Very low levels of the RTVP-1 protein were observed in LGA and higher levels were detected in GBM (FIG. 8e). These results indicate increased levels of RTVP-1 mRNA GBM result in increased levels of RTVP-1 protein.

In summary, these results confirm that RTVP-1 is preferentially expressed in GBM and may therefore serve as a reliable diagnostic marker for gliomas and suggest potential clinical utility of RTVP-1 as a molecular marker indicative of tumor grade.

### Example 3

### Silencing of RTVP-1 Gene in Gliomas Induces Apoptosis and Reduces Cell Proliferation

Recent studies (Scherr et al. (2003) Curr. Med. Chem. 10:245-256) indicate that gene silencing can be obtained by using 21-23 nucleotide double-stranded RNA (siRNA). This strategy is known as RNA interference (RNAi).

In order to examine the effect of destabilizing of an RTVP-1 transcript in gliomas, two different siRNAs to silence RTVP-1 gene expression were prepared (siRNA1:SEQ. ID. NOs.: 1 and 2 and siRNA2: SEQ. ID. NOs.: 3 and 4).

In an initial experiment the U87 and A172 glioma cell limes were transfected with siRNA1. RTVP-1 expression was assayed by RT-PCR as described hereinabove after 72 hr.

FIGS. 1a and 1b show that transfection of the cells with siRNA1 decreased RTVP-1 mRNA in both cell lines following 72 hr. A negative control siRNA (siRNA: SEQ. ID. NOs.: 5 and 6) had no effect. Similar results were obtained in a prostate cancer cell line, PC-3.

In order to examine the role of RTVP-1 in glioma cell proliferation cells were transfected with RTVP-1 siRNA and with control siRNA as detailed hereinabove. Expression of Proliferating Cell Nuclear Antigen (PCNA) was performed by Western blot analysis using a commercially available rabbit polyclonal primary antibody (Santa Cruz Biotechnology; Santa Cruz Calif.; USA) in parallel with counting of cells treated with control siRNA or RTVP-siRNA.

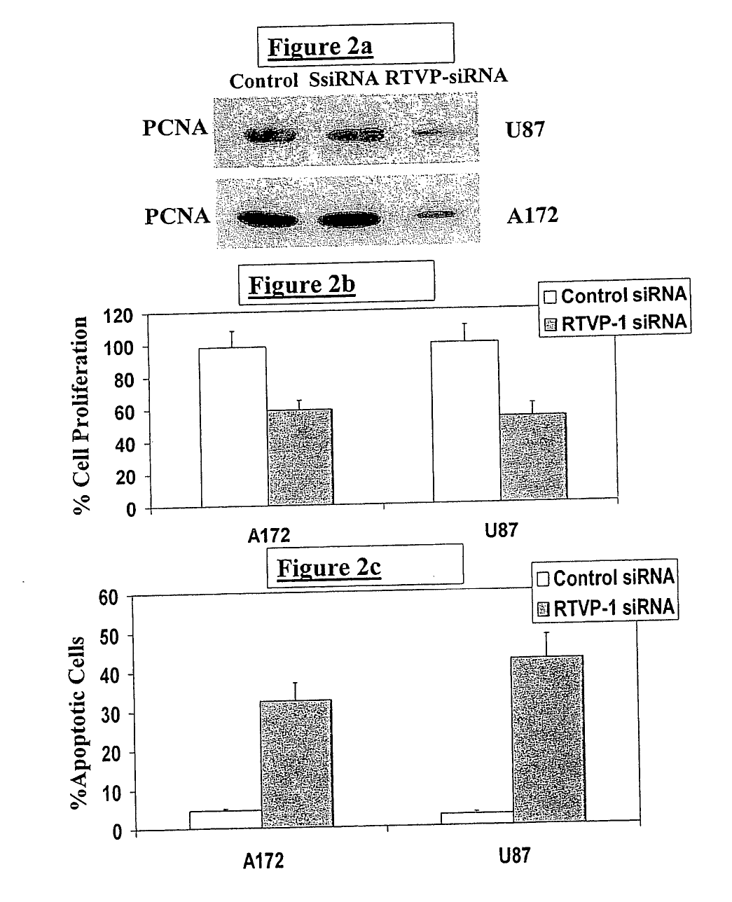

The silencing of RTVP-1 decreased the expression of PCNA (a marker of cell proliferation, see FIG. 2a) and glioma cell number (FIG. 2b) in the U87 and the A172 glioma cell lines. In addition, silencing of RTVP-1 induced cell apoptosis (FIG. 2c) in both the U87 and A172 cells. These effects were first observed after 48 hr of transfection and reached plateau levels after 72 hr (FIGS. 2a, 2b and 2c summarize results from 72 hours).

In an additional experiment, a second siRNA (SiRTVP-1(II): SEQ ID NOs.: 3 and 4) directed against RTVP-1 mRNA was assayed in the U87 and the U251 glioma cell lines. Results were similar to those described above and are summarized in FIGS. (3a, 3b , and 3c ). The second siRNA duplex significantly reduced the level of RTVP-1 as determined by RT-

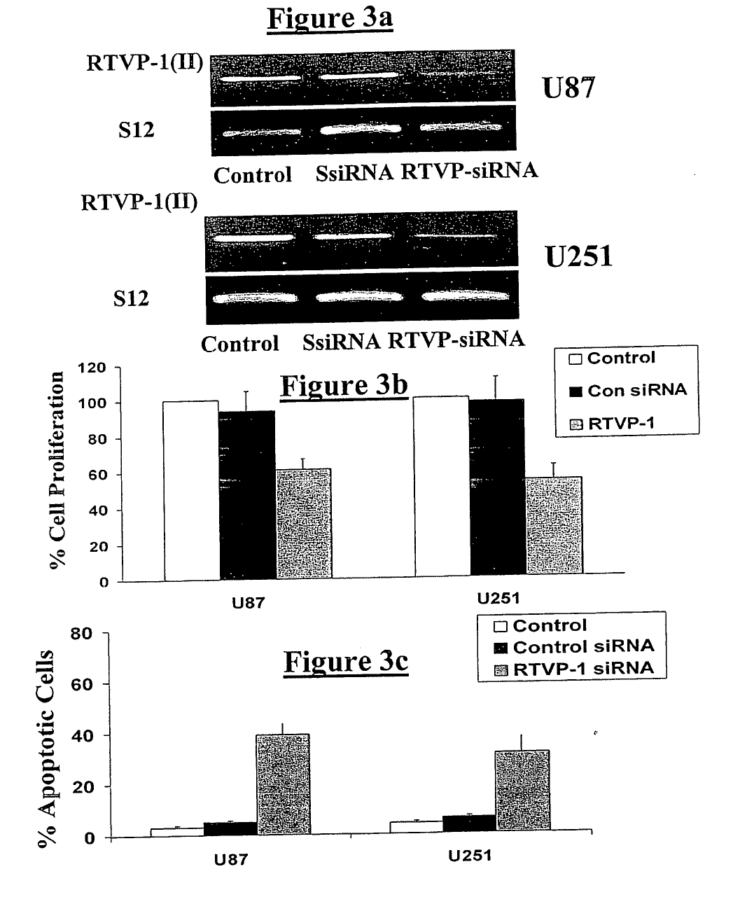

PCR (FIG. 3a) while a control scrambled siRNA had no effect. Cells transfected with the siRNA-RTVP-1 (II) exhibited reduced cell proliferation and increased cell apoptosis (FIGS. 3b and 3c ).

In order to increase the duration of siRNA effect, shRNA vectors which allow optimal expression of siRNA in a wide variety of cell types and permit stable down regulation of RTVP were prepared. Vectors expressing the two shRNA sequences corresponding to the siRNAs described hereinabove were prepared.

Briefly, pRNA-U6.1/Neo Genscript (Piscataway, N.J.) is a GenScript siRNA expression vector. It is designed for mammalian transfection. It carries a Neomycin resistance gene which can be used for establishing stable cell lines and has a U6 promoter for the expression of the shRNAs. Annealing of SEQ. ID. NOs.: 26 and 27 produced shRNA1 and annealing of SEQ. ID. Nos: 28 and 29 produced shRNA2. shRNA1 and shRNA2 were each independently cloned into BamHI and HindIII digested pRNA-U6.1/Neo to produce pRNART1 and pRNART2 respectively. A commercially available pRNA-U6.1/Neo negative control vector was also employed (Genscript; Piscataway, N.J.).

U87 cells were transfected with the shiRNA plasmid vectors (pRNART1 and pRNART2) using nucleofection (Amaxa GmbH, Cologne; Germany) and RTVP-1 expression was assayed after 72 hr using RT-PCR. A negative control siRNA (PRNACV) was employed as detailed hereinabove.

Data presented in FIG. 4a illustrates that the two plasmids reduced the expression of RTVP-1 in U87 cells as determined by RT-PCR, albeit to a lower degree than naked siRNA duplexes. As expected proliferation decreased (FIG. 4b) and apoptosis increased (FIG. 4c) in cells transfected with the pRNART1 and pRNART2 vectors.

These results indicate that viral shRNA vectors (e.g. adenovirus or lentivirus) vectors may be employed for in vivo delivery of siRNA.

In summary, these results demonstrate that the siRNAs examined were efficient in the silencing of the RTVP-1 mRNA in both glioma and prostate cancer cell lines. Further; these results establish that down regulation of RTVP-1 expression decreased cell proliferation and increased the apoptosis of glioma cells.

### Example 4

### Cloning of the Human RTVP-1 Promoter and Preparation of Reporter Gene Constructs

In order to provide a glioma specific promoter for use in gene therapy vectors a 1951-bp fragment (designated E in FIG. 9; SEQ. ID. NO.: 25) of the RTVP promoter was amplified by PCR from isolated BCBL-1 cells genomic DNA using synthetic oligonucleotide primers derived from a published human genomic sequence (Homo sapiens chromosome 12 genomic contig VERSION NT_{—}029419.10 GI:29803948): Pro #235 5′-GCA CGC GTG TTT GTT TGG TTG GTT GGT TG-3′ (bases-1972_-1951: SEQ. ID. NO.: 15); and Pro #183 5′-TAA CTC GAG ATG CTT TGC TGG CT-3′ (bases +1-14: SEQ. 10. NO.: 16) (the restriction sites in bold). The resulting PCR product was eluted from low-melting point agarose, purified, excised with MIul and YhoI restriction enzymes and cloned into the same sites of vector pGL3-Basic (Promega, Wis., USA) containing the luciferase reporter gene to yield pGL3-1951 (containing fragment E in FIG. 9).

Deletions from the 5′ and 3′ end of the “E” fragment were made by PCR amplification of pGL3-1951 using internal RTVP promoter primers having an MluI site at the 5′ end, and XhoI site at the 3′ end. Digesting the PCR product with MluI and XhoI, and cloning into MluI XhoI-digested pGL3-basic. A schematic comparison of resultant fragments A, B, C and D is shown in FIG. 9. In each cases, the fragments reside upstream of the Luciferase reporter gene in the correct orientation.

A 1439-bp fragment of the RTVP promoter (D in FIG. 9) was amplified by PCR using synthetic oligonucleotide primers: Pro #219 5′-GCA CGC GTC TCT CTT AAT TTT CTA AAA TAC ACG-3′ (bases-1439_-1414: SEQ. ID. NO.: 17); and Pro #183 5′-TAA CTC GAG ATG CTI TGC TGG CT-3′ (bases +1-14: SEQ. 10. NO.: 16) to yield pGL3-1439.

A 1311-bp fragment of the RTVP promoter (C in FIG. 9) was amplified by PCR using synthetic oligonucleotide primers: Pro #219 5′-GCA CGC GTC TCT CTT AAT TTT CTA AAA TAC ACG-3′ (bases-1439_-1414: SEQ. ID. NO.: 17); and Pro #220 5′-GCC TCG AGC AGA ACA GAG CAT GAG TTC ATC ACT A-3′ (bases-128_-149: SEQ. ID. NO.: 18) to yield pGL.3-1439_-128.

A 913-bp fragment of the RTVP promoter (B in FIG. 9) was amplified by PCR using synthetic oligonucleotide primers: Pro #227 5′-ACA CGC GTC AGC CCC TGT TGT AAC ATC CT-3′ (bases-913_-892: SEQ. ID. NO.: 19); and Pro #183 5′-TAA CTC GAG ATG CTT TGC TGG CT-3′ (bases +1-14: SEQ. ID. NO.: 16) to yield pGL.3-1439.

A 338-bp fragment of the RTVP promoter (A in FIG. 9) was amplified by PCR using synthetic oligonucleotide primers: Pro #236 5′-GCA CGC GTC CAG ATA TTC CAA CCA CTA TGT GT-3′ (bases-338_-314: SEQ. ID. NO.: 17); and Pro #220 5′-GCC TCG AGC AGA ACA GAG CAT GAG TTC ATC ACT A-3′ (bases-128-149: SEQ. ID. NO.: 18) to yield pGL3-1439_-128.

### Example 5

### Analysis of RTVP-1 Promoter using a Reporter Gene

Constructs described in Example 4 were subsequently used to transfect cells in order to determine what portions of the largest fragment (E) were responsible for promoter activity. Transfection and Luciferase assay were conducted as described hereinabove in Materials and Methods.

In an initial experiment, the human U87 glioma cells were co-transfected with two reporter plasmids: one containing firefly-luciferase under the putative RTVP-1 promoter regulatory elements (Fragments C and D, see FIG. 9) and the other one containing renila luciferase under the SV40 promoter (Promega). An empty vector containing the luciferase gene but no promoter fragment was employed as a negative control. Cells were collected 48 hr after transfection and analyzed using the dual luciferase kit (Promega). Relative activity was calculated as 100× Firefly luciferase renilla-luciferase. Results are summarized graphically in FIG. 10a. Fragment D had significantly higher promoter activity as compared with fragment C. Similar results were obtained in three independent experiments.

In a subsequent experiment, the transcriptional activity of Fragment D was compared in HeLa cells that do not express RTVP-1 and U87 cells (FIG. 10b). As expected, the promoter activity of Fragment D was specific to cells of glioma origin and no activity was observed in the HeLa cells that do not express RTVP-1.

In order to determine the optimal promoter fragment, plasmids containing fragments A,B,C,D and E were each transfected into the U87 cells as described hereinabove. Fragment D produced the highest Luciferase activity The lower promoter activity of fragment E with respect to fragment D indicates that a negative regulatory element apparently resides between (−) 1439 and (−) 1951. A positive regulatory element seems to reside between (0) and (−) 130 as evidenced by the higher activity of fragments Band D with respect to fragment C. Surprisingly, about half of the total positive regulatory activity of the strongest fragment (D) is retained by the shortest fragment (A; (−) 130 to (−) 466).

In summary, the human RTVP-1 promoter fragments A, B,C,D and E (SEQ. ID. NOs.: 21-25 respectively) all contain significant promoter activity which appears specific to glioma cells Use of these promoters in cell type specific gene therapy and in creation of cellular and/or animal models for glioma appears feasible. Previously available alternatives did not allow specific targeting of therapeutic agents to glioma cells only. Thus, the present invention provides the possibility of glioma specific targeting for the first time.

### Example 6

### Predictive Value of RTVP-1 mRNA Level

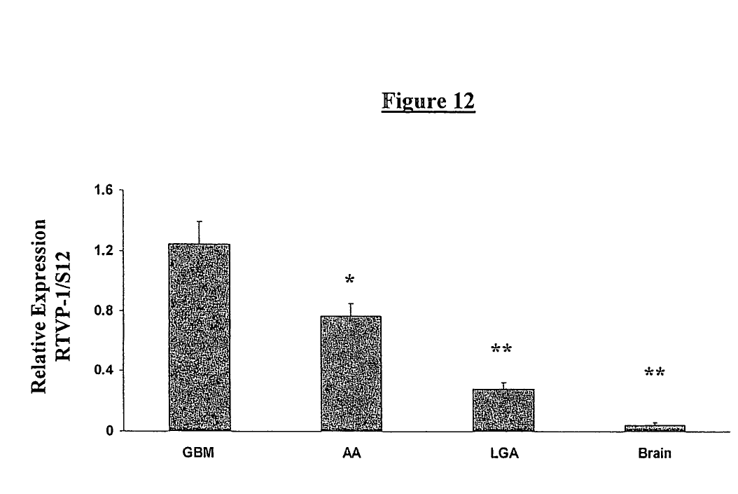

In order to demonstrate the utility of RTVP-1 mRNA level in the context of glial cell transformation, data presented in FIGS. 6a; 6b; 6c; 7a; 7b; 8a; 8b; 8c; and 8d were quantified as detailed hereinabove and analyzed using one way ANOVA. The level of significance of observed differences between expression levels in GBM, LGA, AA and normal brain was determined. Results are presented graphically in FIG. 12. Each bar represents the relative expression of RTVP-1 normalized to S12 mRNA as determined by RT-PCR as described hereinabove. The results are presented as the mean values ±S.E. (*p<0.001; ** p<0.002; relative to GBM). These results indicate that RTVP-1 transcript levels have predictive value in identifying patients that are likely to undergo the critical transformation between LGA and AA. According to these results, patients with RTVP-1 transcript levels above the mean for LGA should be treated aggressively. Treatment might be based upon, for example, gene therapy protocol employing promoter fragment E (SEQ. ID. NO.: 25) or a functional portion thereof as detailed hereinabove. Alternately, or additionally, treatment might be based upon, for example, a siRNA (e.g. siRNA1 (SEQ. ID. NOs.: 5 and 6); siRNA II (SEQ. ID. NOs.: 3 and 4)) or an shRNA (e.g. shRNA1 (SEQ. ID. NOs.: 26 and 27 or shRAN2 (SEQ. ID. Nos: 28 and 29)).

Further, these results indicate that it is both feasible and desirable to provide standards for calibration to be employed in conjunction with a diagnostic kit as described hereinabove. Use of such calibration standards will facilitate reliable prognosis of likelihood of glial transformation for individual patients.

### Example 7

### Demonstration that RTVP-1 is a Secreted Protein

In order to demonstrate the possibility of using RTVP-1 as a marker to monitor tumor status or progression without resort to invasive biopsies, a preliminary experiment in cell culture was conducted. The FLAG™ expression tag was employed (Stratagene; La Jolla Calif.; USA). Briefly, U87 cells were transfected with either an expression vector containing RTVP-I in pCMVtg2b or with the empty pCMVtg2b vector (CV) for 24 hr. Medium was changed to serum free medium and after additional 24 hr, cells and cell supernatants were collected, processed and analyzed by Western blot analysis.

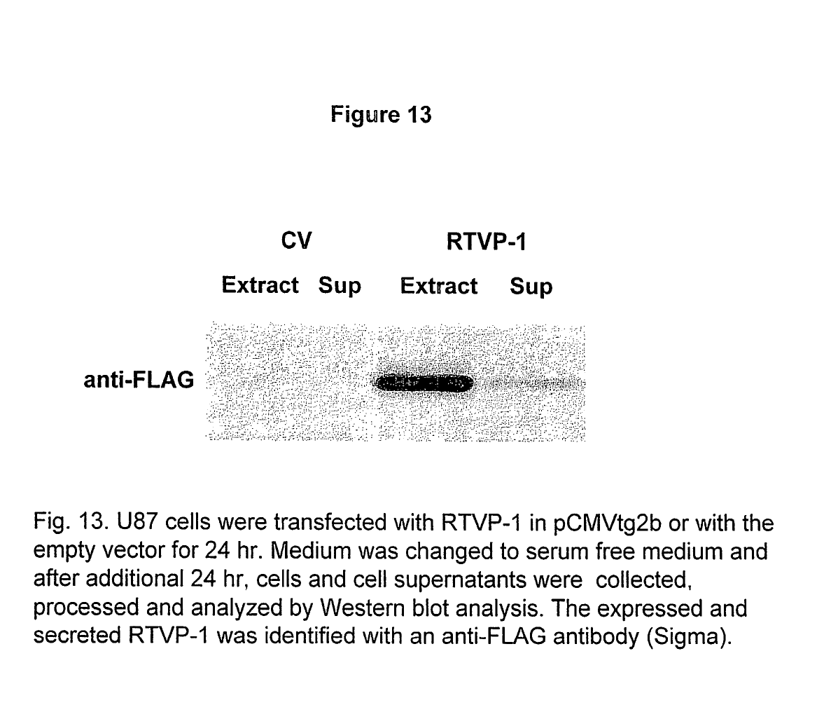

As expected, the immuno-blot (FIG. 13) clearly illustrates the presence of a FLAG™ expression tag in whole cell extract as determined by an anti-FLAG™ primary antibody. Surprisingly, culture media from cells transfected with the expression vector containing RTVP-1 also contained detectable amounts of FLAG™ expression tag (FIG. 13).

This result is the first report of RTVP-1 secretion and suggests that monitoring of RTVP-1 levels in a subject may be conducted by assaying cerebrospinal fluid, or perhaps even peripheral blood, without resort to more invasive biopsies of solid tissue.

### Example 8

### Expression of RTVP-1 in Astrocytic Tumors with Different Degree of Malignancy

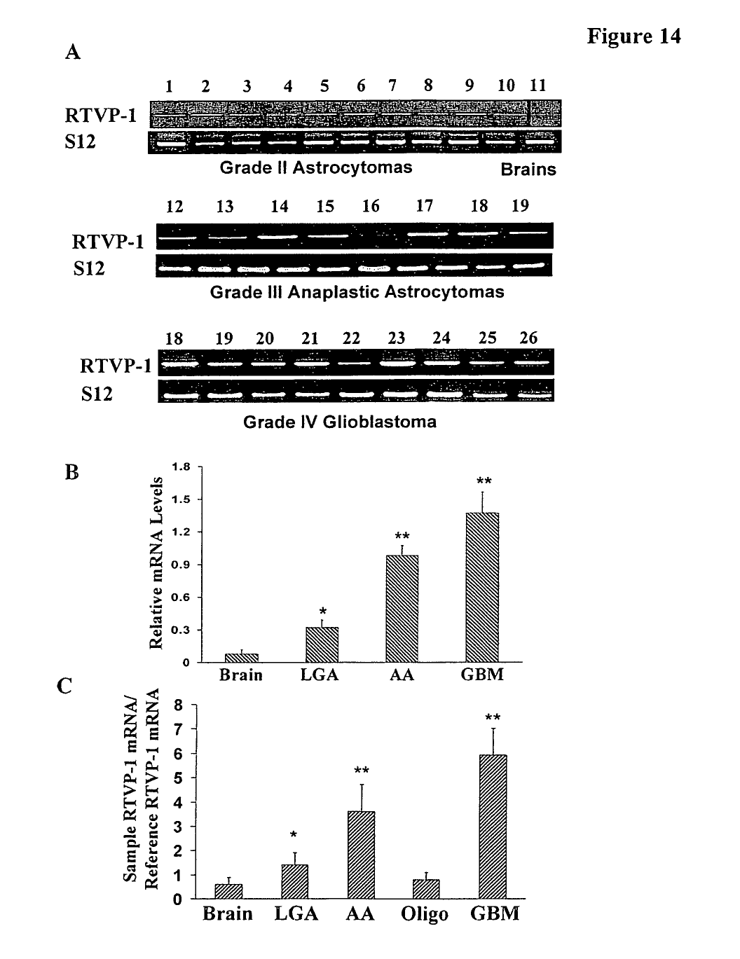

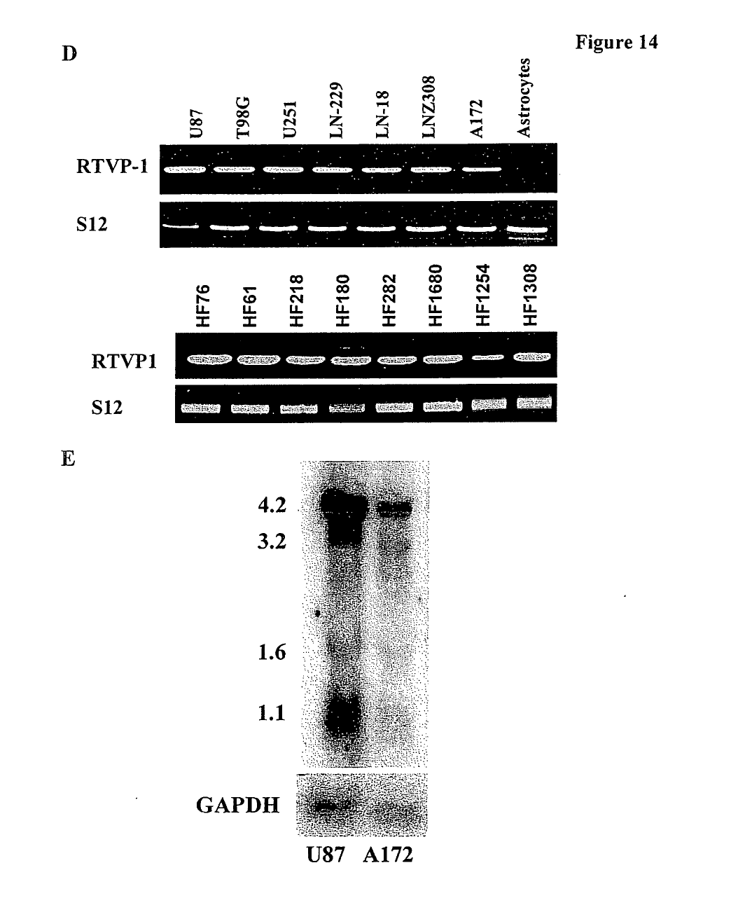

RTVP-1 has been previously reported to be expressed in glioblastomas, however its expression in other types of astrocytic tumors has not been reported. To characterize the expression of RTVP-1 in different types of astrocytic tumors, we employed tumor samples from low-grade astrocytomas (LGA, grade II), anaplastic astrocytomas (AA, grade III) and glioblastomas (GBM, grade IV). The expression of RTVP-1 in these tumors was compared to that of normal brains using semi-quantitative RT-PCR. As presented in FIG. 14a, the expression of RTVP-1 in normal brains was barely detected. Similarly, most low-grade astrocytomas expressed very low levels of RTVP-1. In contrast, higher levels of RTVP-1 were observed in anaplastic astrocytomas and glioblastomas (FIG. 14a). Quantitation of RTVP-1 expression as compared to that of S12 clearly demonstrates that the expression of RTVP-1 is significantly higher in GBM and AA as compared to LGA and normal brains (FIG. 14b).

Similar results were obtained using real-time RT-PCR (FIG. 14c). Thus, the relative RTVP-1 mRNA expression to S12 in brains and low-grade astrocytomas was very low (0.6±0.28 and 1.1±0.54, respectively), whereas glioblastomas expressed significantly higher levels of RTVP-1 (5.9±1.1). Interestingly, the expression of RTVP-1 was very low in oligodendroglioma (0.8±0.21), both low-grade and anaplastic). Thus, the expression of RTVP-1 can distinguish between anaplastic astrocytomas and anaplastic oliogodendrogliomas.

The expression of RTVP-1 was also examined in glioma cell lines and primary glioma cultures. As presented in FIG. 14d, RTVP-1 was expressed in all the glioma cells examined, whereas it was barely detected in human fetal astrocytes (FIG. 14d)

Using Northern blot analysis, we observed a strong expression of the 4.2, 3.2 and 1.1 kb transcripts in the U87 and A 172 glioma cell lines and a weaker expression of the 1.6 kb transcript in these cells (FIG. 14e).

Previous studies reported the expression of four distinct mRNAs of 4.2, 3.2, 1.6 and 1.0 kb by using an RTVP-1 DNA probe. To further explore this point, we examined the effect of the RTVP-1 siRNA on the expression of the different mRNAs. We found that the RTVP-1 siRNA which targets the first exon of RTVP-1, significantly reduced the expression of the 1.6 and 1.0 kb mRNAs, whereas it did not significantly affect the expression of the 4.2 and 3.2 transcripts (data not shown).

### Example 9

### Silencing of RTVP-1 Decreases Cell Proliferation and Induces Cell Apoptosis

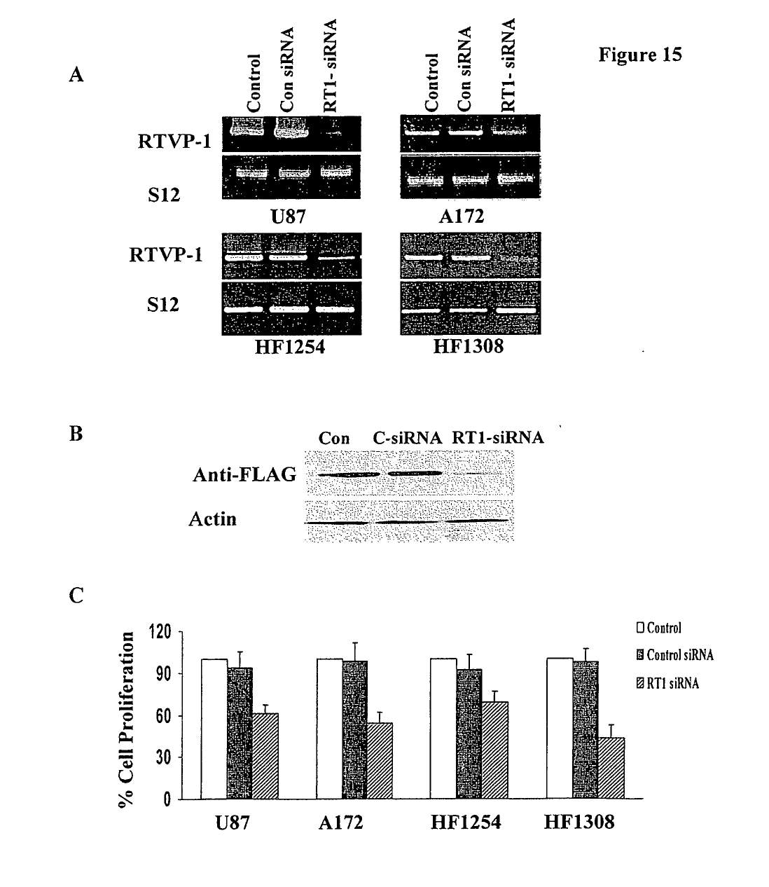

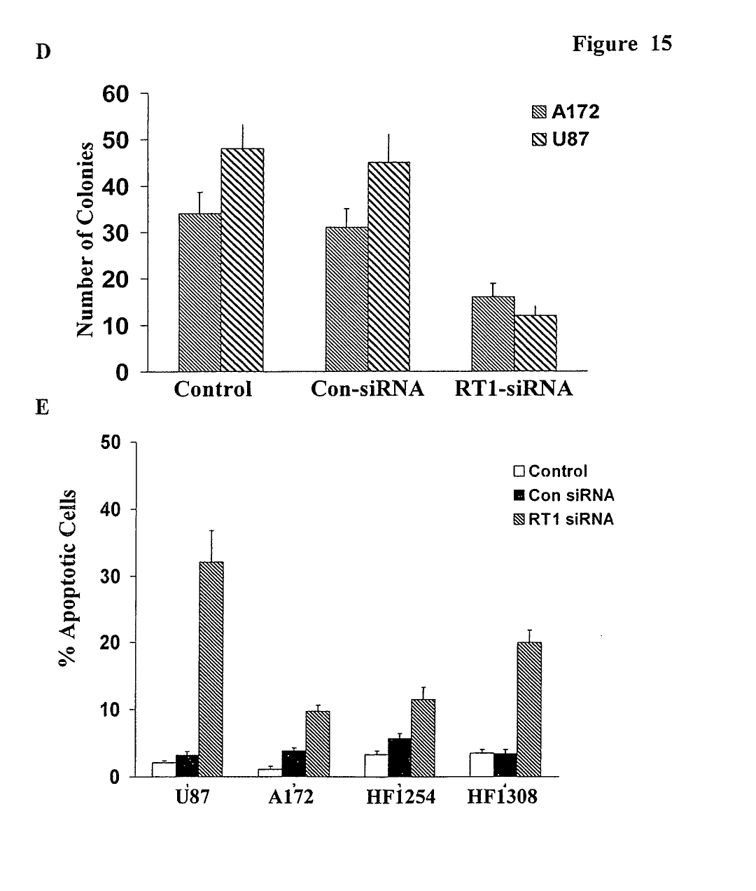

To examine the role of RTVP-1 in glioma cell function, we first employed siRNAs directed against the RTVP-1 mRNA. Transfection of the glioma cell lines, U87 and A172 with a siRNA duplex for three days resulted in around 90% and 70% decrease in the expression of the RTVP-1 mRNA, respectively, whereas no decrease in the expression of RTVP-1 was observed with a scrambled control siRNA (FIG. 15a). The RTVP-1 siRNA also decreased the expression of the RTVP-1 protein as represented in FIG. 3B. Transfection of U87 (FIG. 15b) and A172 cells (data not shown) overexpressing RTVP-1 with RTVP-1 siRNA significantly decreased the expression of RTVP-1 after 72 hr as detected by anti-FLAG antibody, whereas a control siRNA did not have a significant effect.

The RTVP-1 siRNA transfected U87 cells exhibited a significant decrease in cell proliferation (FIG. 15c) and in cell growth in soft agar (FIG. 15d). In addition, about 30% of the U87 cells were apoptotic as determined by PI staining and FACS analysis (FIG. 15e). The A172 cells, in which RTVP-1 expression was less reduced by the siRNAs, exhibited a significant decrease in cell proliferation (FIG. 15c) and cell growth in soft agar (FIG. 15d) but a smaller degree of cell apoptosis (FIG. 15e). Similar results were observed with another set of RTVP-1 duplex (data not shown).

To further examine the effect of RTVP-1 silencing, we employed established primary glioma cultures. Transfection of the primary cultures with RTVP-1 siRNAs reduced the expression of RTVP-1 to a different degree (FIG. 15a). Similar to the results obtained with the glioma cell lines, cells in which RTVP-1 was significantly depleted (HF1308) exhibited both a decrease in cell number (FIG. 15c) and an increase in cell apoptosis (FIG. 15e). In contrast, cells in which RTVP-1 expression was only partially reduced (HF1254), showed a similar decrease in cell number but only a small increase in cell apoptosis (FIGS. 15c, 15e).

### Example 10

### Specificity of the RTVP-1 Promoter

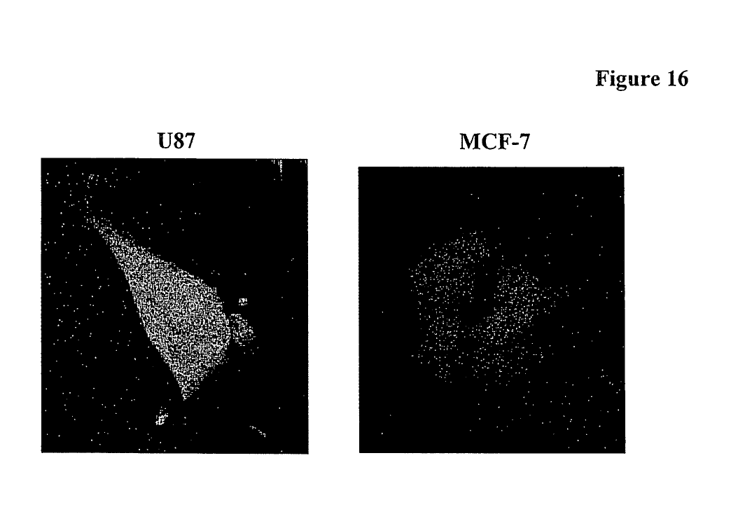

To examine the specificity of the RTVP-1 promoter we first cloned the C fragment into a promoterless EGFP plasmid and then cloned it into the AdEasy promoterless pshuttle vector. We then generated adenovirus vectors that express the GFP protein under the RTVP-1 promoter (fragment C). To examine the specificity of the promoter to cells that express RTVP-1, we infected the U87 glioma cells (that express high levels of RTVP-1) and the MCF-7 breast cancer cells (that express very low levels of RTVP-1) with the GFP-RTVP-1 promoter adenovirus vector. As presented in FIG. 16, the GFP was strongly expressed in the U87 cells, whereas only very low expression was observed in the MCF-7 cells, suggesting that the promoter is selectively expressed in cells that express high levels of RTVP-1.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims. All publications, patents and patent applications mentioned in this specification are herein incorporated in their entirety by reference into the specification, to the same extent as if each individual publication, patent or patent application was specifically and individually indicated to be incorporated herein by reference In addition, citation or identification of any reference in this application shall not be construed as an admission that such reference is available as prior art to the present invention.

What is claimed is:

1. A method of diagnosing glioma in a subject, the method comprising conducting a quantitative analysis of an RTVP expression level in a biological sample taken from the subject.

2. The method of claim 1, wherein said quantitative analysis relies upon quantification of at least a portion of an RTVP mRNA transcript.

3. The method of claim 2, wherein said quantification of at least a portion of an RTVP mRNA transcript is accomplished by RT-PCR.

4. The method of claim 3, wherein said RT-PCR employs at least one primer selected from the group consisting of SEQ. ID. NOs.: 7; 9 and 11.

5. The method of claim 3, wherein said RT-PCR employs at least one primer selected from the group consisting of SEQ. ID. NOs.: 8; 10 and 12.

6. The method of claim 2, wherein said quantification includes a comparison to at least a portion of at least one additional transcript.

7. The method of claim 6, wherein said at least one additional transcript includes an S12 transcript.

8. The method of claim 1, wherein said quantitative analysis of an RTVP expression level in a biological sample taken from the subject employs an antibody specific to at least a portion of an RTVP-1 protein.

9. A diagnostic kit for analysis of a biological sample removed from a subject, the kit comprising:
(a) reagents suitable for conducting a quantitative analysis of an RTVP expression level in the biological sample.

10. The diagnostic kit of claim 9, further comprising
(b) packaging material; and
(c) instructions for performance of said quantitative analysis on at least one type of biological sample.

11. The diagnostic kit of claim 9, wherein said quantitative analysis relies upon quantification of at least a portion of an RTVP mRNA transcript.

12. The diagnostic kit of claim 11, wherein said quantification of at least a portion of an RTVP mRNA transcript is accomplished by RT-PCR.

13. The diagnostic kit of claim 12, wherein said RT-PCR employs at least one primer selected from the group consisting of SEQ. ]D. NOs.: 7; 9 and 11.

14. The diagnostic kit of claim 12, wherein said RT-PCR employs at least one primer selected from the group consisting of SEQ. ID. NOs.: 8; 10 and 12.

15. The diagnostic kit of claim 11, wherein said quantification includes a comparison to at least a portion of at least one additional transcript.

16. The diagnostic kit of claim 15, wherein said at least one additional transcript includes an S12 transcript.

17. The diagnostic kit of claim 10, wherein said instructions further include an explanation of at least one method for collection of the biological sample.

18. The diagnostic kit of claim 10, wherein said instructions further include an explanation of diagnosing glioma in the subject based upon a result of the analysis.

19. The diagnostic kit of claim 9, wherein said quantitative analysis of an RTVP expression level in a biological sample taken from the subject employs an antibody specific to at least a portion of an RTVP-1 protein.

20. A method of influencing a clinical progression of glioma in a subject, the method comprising reducing a biological activity of RTVP-1 in the subject.

21. The method of claim 20, wherein said reducing of said biological activity is accomplished by influencing at least one item selected from the group consisting of a level of transcription of an RTVP-1 gene, a stability of an RTVP-1 mRNA transcript, a level of translation of an RTVP-1 mRNA transcript, a level of activity of an RTVP-1 protein and a stability of an RTVP-1 protein.

22. The method of claim 21, wherein said influencing a stability of an RTVP-1 mRNA transcript is accomplished by administration of siRNA to the subject.

23. The method of claim 21, wherein said siRNA includes at least one pair of complementary RNA sequences selected from the group consisting of SEQ. ID. NOs: 1 and 2; SEQ. ID. NOs: 3 and 4; SEQ. ID. NOs: 31 and 32; SEQ. ID. NOs: 26 and 27; and SEQ. ID. NOs: 28 and 29.

24. The method of claim 20, wherein said reducing biological activity of RTVP-1 employs an antibody specific to at least a portion of an RTVP-1 protein.

25. A pharmaceutical composition for treating glioma, the pharmaceutical composition comprising as an active ingredient a physiologically effective amount of an agent which reduces a biological activity of RTVP-1 in a subject treated with the pharmaceutical composition and a physiologically acceptable carrier and excipient.

26. The pharmaceutical composition of claim 25, wherein said agent which reduces a biological activity acts by influencing at least one item selected from the group consisting of a level of transcription of an RTVP-1 gene, a stability of an RTVP-1 mRNA transcript, a level of translation of an RTVP-1 mRNA transcript, a level of activity of an RTVP-1 protein and a stability of an RTVP-1 protein.

27. The pharmaceutical composition of claim 26, wherein said influencing a stability of an RTVP-1 mRNA transcript is accomplished by administration of siRNA to the subject.

28. The pharmaceutical composition of claim 27, wherein said siRNA includes at least one pair of complementary RNA sequences selected from the group consisting of SEQ. ID. NOs: 1 and 2; SEQ. ID. NOs: 3 and 4; SEQ. ID. NOs: 31 and 32; SEQ. ID. NOs: 26 and 27; and SEQ. ID. NOs: 28 and 29.

29. The pharmaceutical composition of claim 25, wherein said agent which reduces a biological activity of RTVP-1 from the subject employs an antibody specific to at least a portion of an RTVP-1 protein.

30. A method of formulation of a pharmaceutical composition for treatment of glioma, the method comprising combining an agent which reduces a biological activity of RTVP-1 with a physiologically acceptable carrier and excipient.

31. The method of claim 30, wherein said agent which reduces a biological activity acts by influencing at least one item selected from the group consisting of a level of transcription of an RTVP-1 gene, a stability of an RTVP-1 mRNA transcript, a level of translation of an RTVP-1 mRNA transcript, a level of activity of an RTVP-1 protein and a stability of an RTVP-1 protein.

32. The method of claim of claim 31, wherein said influencing a stability of an RTVP-1 mRNA transcript is accomplished by said agent in the form of an siRNA.

33. The method of claim of claim 32, wherein said siRNA includes at least one pair of complementary RNA sequences selected from the group consisting of SEQ. ID. NOs: 1 and 2; SEQ. ID. NOs: 3 and 4; SEQ. ID. NOs: 31 and 32; SEQ. ID. NOs: 26 and 27; and SEQ. ID. NOs: 28 and 29.

34. The method of claim 30, wherein said agent which reduces a biological activity of RTVP-1 employs an antibody specific to at least a portion of an RTVP-1 protein.

35. An isolated nucleic acid sequence characterized by an ability to positively regulate a downstream gene, the sequence comprising an item selected from the group consisting of SEQ. ID. NO.: 25 and a functional portion thereof.

36. The isolated nucleic acid sequence of claim 35, wherein said functional portion thereof is selected from the group consisting of SEQ. ID, NOs.: 21; 22; 23 and 24.

37. An expression vector comprising the isolated nucleic acid sequence of claim 35 and said downstream gene.

38. A mammalian cell transfected with an expression vector according to claim 37.

39. A transgenic animal, the transgenic animal comprising at least one exogenous copy of the isolated nucleic acid sequence of claim 35 within a genome thereof.
